(19) United States
(12) Patent Application Publication
(10) Pub. No.: US 2024/0279175 A1
(43) Pub. Date: Aug. 22, 2024

(54) CRYSTALLINE HYDROCHLORIDE SALTS OF SUBSTITUTED TRYPTAMINES

(71) Applicant: CAAMTECH, INC., Issaquah, WA (US)

(72) Inventor: Andrew R. CHADEAYNE, Issaquah, WA (US)

(21) Appl. No.: 18/573,601

(22) PCT Filed: Jun. 28, 2022
(86) PCT No.: PCT/US2022/035262

§ 371 Date: Dec. 22, 2023

Related U.S. Application Data
(60) Provisional application No. 63/216,159, filed on Jun. 29, 2021.

(51) Int. Cl. C07D 209/16; A61K 31/4045; A61K 45/06

(52) U.S. Cl. CPC C07D 209/16; A61K 31/4045; A61K 45/06

(57) ABSTRACT

The disclosure relates to substituted tryptammonium hydrochloride salts, crystalline substituted tryptammonium hydrochloride salts, and specific crystalline forms thereof, including crystalline forms 1 of a substituted tryptammonium hydrochloride salt of the disclosure, to compositions containing the same, and to methods of treatment using them.

### CROSS REFERENCE TO RELATED APPLICATIONS

This application claims priority to U.S. Provisional Application No. 63/216,159, filed on Jun. 29, 2021, the disclosure of which is incorporated by reference.

### TECHNICAL FIELD

This disclosure relates to hydrochloride salts of substituted tryptamine derivatives, crystalline hydrochloride salts of substituted tryptamine derivatives, and specific crystalline forms thereof, including crystalline form 1 of hydrochloride salts of substituted tryptamine derivatives; to pharmaceutical compositions containing hydrochloride salts of substituted tryptamine derivatives or crystalline hydrochloride salts of substituted tryptamine derivatives, including crystalline form 1 of hydrochloride salts of substituted tryptamine derivatives; and to methods of treatment/therapeutic uses of hydrochloride salts of substituted tryptamine derivatives or crystalline hydrochloride salts of substituted tryptamine derivatives, including crystalline form 1 of hydrochloride salts of substituted tryptamine derivatives.

### BACKGROUND OF THE INVENTION

Tryptamine is an indolealkylamine that is the metabolite of the tryptophan, one of the essential amino acids that humans obtain through their diet. It can be found in high quantities in the gastrointestinal tract, and plays a role in regulating electrolyte balance. The structure of tryptamine is the core of many neuromodulators, including melatonin and serotonin. It is also the basis for naturally occurring psychedelics found in ayahuasca (DMT), toads (bufotenine), and magic mushrooms (psilocybin/psilocin). These and similar dialkyltryptamines have garnered a great deal of interest due to their potential in treating mental disorders including addiction, anxiety, depression, and post-traumatic stress disorder. Varying the substitution pattern on the indole of tryptamines can greatly impact the activity of the compound as a neuromodulator. The simple addition of hydroxide at the 5 position of tryptamine generates serotonin, the key hormone in regulating mood. Other variations can change the receptor profile of a tryptamine, changing both the receptors at which it is active, and the degree of activity. Changing the indole hydrogens to alkyl, halo, and alkoxy groups at positions 4-7 alters the ability of the compound to act as an agonist at the serotonin receptors (5-hydroxytyptamine, 5-HT), with studies showing changes at 5-HT_{1A}, 5-HT_{1D}, and 5-HT_{2A}, as well as other important receptors including the serotonin transporter (SERT) and the N-methyl-D-aspartate (NMDA) receptors (Berger et al., 2012; Chang et al., 1993; Peroutka et al., 1991).

Tryptamine based pharmaceuticals are already widely used in humans, with migraine drugs including sumatriptan having been prescribed for 30 years. With the recent designation by the United States Food and Drug Administration of psilocybin as a “breakthrough therapy”, the expectation is that tryptamine-based serotonin 2A agonists will continue to grow as mood disorder treatments. As these studies continue, understanding the structure activity relationship of the substitution on the indole ring (as well as with nitrogen alkylation) is going to be critical to understand activity and in the design of improved pharmaceuticals. To this end, there is a need to obtain the structural data for substituted tryptamines, which are reported herein as their hydrochloride salts.

### SUMMARY OF THE INVENTION

This disclosure relates to the following hydrochloride salts of substituted tryptamine derivatives (the “tryptammonium compounds of the disclosure”), crystalline tryptammonium compounds of the disclosure, and specific crystalline forms thereof:
2-(7-methyl-1H-indol-3-yl)ethan-1-aminium chloride (1-methyltryptammonium chloride or 1-Me-T·HCl);
2-(2-methyl-1-phenyl-1H-indol-3-yl)ethan-1-aminium chloride (1-phenyl-2-methyltryptammonium chloride or 1-Ph-2-Me-T·HCl);
2-(5-methoxy-1H-indol-3-yl)ethan-1-aminium chloride (5-methoxytryptammonium chloride or 5-MeO-T·HCl);
2-(5-bromo-1H-indol-3-yl)ethylazanium chloride (5-bromotryptammonium chloride or 5-Br-T·HCl);
2-(5-chloro-1H-indol-3-yl)ethylazanium chloride (5-chlorotryptammonium chloride or 5-Cl-T·HCl);
2-(5-fluoro-1H-indol-3-yl)ethan-1-aminium chloride (5-fluorotryptammonium chloride or 5-F-T·HCl);
2-(5-methyl-1H-indol-3-yl)ethylazanium chloride (5-methyltryptammonium chloride or 5-Me-T·HCl);
2-(6-fluoro-1H-indol-3-yl)ethan-1-aminium chloride (6-fluorotryptammonium chloride or 6-F-T·HCl); and
2-(7-methyl-1H-indol-3-yl)ethyl]azanium chloride (7-methyltryptammonium chloride or 7-Me-T·HCl).

The disclosure further relates to a composition comprising an effective amount of a tryptammonium compound of the disclosure, a crystalline tryptammonium compound of the disclosure, or specific crystalline forms thereof, such as crystalline form 1 of a tryptammonium compound of the disclosure, and at least one excipient.

The disclosure also relates to pharmaceutical compositions comprising a therapeutically effective amount of a tryptammonium compound of the disclosure, a crystalline tryptammonium compound of the disclosure, or a specific crystalline form thereof, such as crystalline form 1 of a tryptammonium compound of the disclosure, and a pharmaceutically acceptable excipient.

The disclosure also provides a composition comprising a tryptammonium compound of the disclosure, a crystalline tryptammonium compound of the disclosure, or specific crystalline forms thereof, such as crystalline form 1 of a tryptammonium compound of the disclosure, as a first component and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone; and at least one excipient.

The disclosure also relates to the therapeutic uses of tryptammonium compounds of the disclosure, crystalline tryptammonium compounds of the disclosure, and specific crystalline forms thereof, such as crystalline form 1 of a tryptammonium compound of the disclosure, are described below as well as compositions containing each of them.

The disclosure also relates to a method of preventing or treating a psychological disorder comprising the step of administering to a subject in need thereof a therapeutically effective amount of a tryptammonium compound of the disclosure, a crystalline tryptammonium compound of the disclosure, or specific crystalline forms thereof, such as crystalline form 1 of a tryptammonium compound of the disclosure, or a composition according to this disclosure.

The disclosure further relates to a method of preventing or treating inflammation and/or pain, preventing or treating a neurological disorder, modulating activity of a mitogen activating protein (MAP), modulating neurogenesis, or modulating neurite outgrowth comprising the step of administering to a subject in need thereof a therapeutically effective amount of a tryptammonium compound of the disclosure, a crystalline tryptammonium compound of the disclosure, or specific crystalline forms thereof, such as crystalline form 1 of a tryptammonium compound of the disclosure, and to administering a pharmaceutical composition or a composition according to the invention.

### DESCRIPTION OF THE FIGURES

FIG. 1 depicts the ORTEP molecular structure of the following compounds: (1) crystalline form 1 of 1-methyltryptammonium chloride (1-Me-T·HCl), (2) crystalline form 1 of 1-phenyl-2-methyltryptammonium chloride (1-Ph-2-Me-T·HCl), (3) crystalline form 1 5-methoxytryptammonium chloride (5-MeO-T·HCl), (4) crystalline form 1 of 5-bromotryptammonium chloride (5-Br-T·HCl), (5) crystalline form 1 of 5-chlorotryptammonium chloride (5-Cl-T·HCl), (6) crystalline form 1 of 5-fluorotryptammonium chloride (5-F-T·HCl), (7) crystalline form 1 of 5-methyltryptammonium chloride (5-Me-T·HCl), (8) crystalline form 1 of 6-fluorotryptammonium chloride (6-F-T·HCl), and (9) crystalline form 1 of 7-methyltryptammonium chloride (7-Me-T·HCl), showing the atomic labeling. Displacement ellipsoids are drawn at the 50% probability level. Hydrogen bonds are shown as dashed lines.

FIG. 2 depicts the crystal packing of crystalline form 1 of 1-methyltryptammonium chloride along the c-axis (bottom right), which shows the one-dimensional hydrogen bonding network along [001]. This network consists of R²₄ (18) rings and C¹₂ (4) chains, shown in the figure. Hydrogens not involved in hydrogen bonding have been removed for clarity.

FIG. 3 depicts the crystal packing of crystalline form 1 of 1-phenyl-2-methyltryptammonium chloride along the a-axis (bottom right), which shows the two-dimensional hydrogen bonding network along [100]. This network consists of R⁴₆(12) and R⁴₈(16) rings and C²₄(8) chains, shown in the figure. Hydrogens not involved in hydrogen bonding have been removed for clarity.

FIG. 4 depicts the crystal packing of crystalline form 1 of 5-methoxytryptammonium chloride along the b-axis.

FIG. 5 depicts the crystal packing of crystalline form 1 of 5-bromotryptammonium chloride along the b-axis (bottom right), which shows the two-dimensional hydrogen bonding network along (001). This network consists of R²₄ (8) and R²₄ (18) rings and C¹₂ (4) and C¹₂ (9) chains, shown in the figure. Hydrogens not involved in hydrogen bonding have been removed for clarity.

FIG. 6 depicts the crystal packing of crystalline form 1 of 5-chlorotryptammonium chloride along the c-axis.

FIG. 7 depicts the crystal packing of crystalline form 1 of 5-fluorotryptammonium chloride along the b-axis.

FIG. 8 depicts the crystal packing of crystalline form 1 of 5-methyltryptammonium chloride along the c-axis (bottom right), which shows the two-dimensional hydrogen bonding network along (100). This network consists of R²₄ (8) and R²₄ (18) rings and C¹₂ (4) and C¹₂ (9) chains, shown in the figure. Hydrogens not involved in hydrogen bonding have been removed for clarity.

FIG. 9 depicts the crystal packing of crystalline form 1 of 6-fluorotryptammonium chloride along the b-axis.

FIG. 10 depicts the crystal packing of crystalline form 1 of 7-methyltryptammonium chloride along the b-axis.

FIG. 11 shows a simulated X-ray powder diffraction pattern (XRPD) for crystalline form 1 of 1-methyltryptammonium chloride generated from its single crystal data.

FIG. 12 shows a simulated X-ray powder diffraction pattern (XRPD) for crystalline form 1 of 1-phenyl-2-methyltryptammonium chloride generated from its single crystal data.

FIG. 13 shows a simulated X-ray powder diffraction pattern (XRPD) for crystalline form 1 of 5-methoxytryptammonium chloride generated from its single crystal data.

FIG. 14 shows a simulated X-ray powder diffraction pattern (XRPD) for crystalline form 1 of 5-bromotryptammonium chloride generated from its single crystal data.

FIG. 15 shows a simulated X-ray powder diffraction pattern (XRPD) for crystalline form 1 of 5-chlorotryptammonium chloride generated from its single crystal data.

FIG. 16 shows a simulated X-ray powder diffraction pattern (XRPD) for crystalline form 1 of 5-fluorotryptammonium chloride generated from its single crystal data.

FIG. 17 shows a simulated X-ray powder diffraction pattern (XRPD) for crystalline form 1 of 5-methyltryptammonium chloride generated from its single crystal data.

FIG. 18 shows a simulated X-ray powder diffraction pattern (XRPD) for crystalline form 1 of 6-fluorotryptammonium chloride generated from its single crystal data.

FIG. 19 shows a simulated X-ray powder diffraction pattern (XRPD) for crystalline form 1 of 7-methyltryptammonium chloride generated from its single crystal data.

### DETAILED DESCRIPTION

### Compounds

This disclosure relates to the following hydrochloride salts of substituted tryptamine derivatives (the “tryptammonium compounds of the disclosure”), crystalline tryptammonium compounds of the disclosure, and specific crystalline forms thereof:
2-(7-methyl-1H-indol-3-yl)ethan-1-aminium chloride (1-methyltryptammonium chloride or 1-Me-T·HCl);
2-(2-methyl-1-phenyl-1H-indol-3-yl)ethan-1-aminium chloride (1-phenyl-2-methyltryptammonium chloride or 1-Ph-2-Me-T·HCl);
2-(5-methoxy-1H-indol-3-yl)ethan-1-aminium chloride (5-methoxytryptammonium chloride or 5-MeO-T·HCl);
2-(5-bromo-1H-indol-3-yl)ethylazanium chloride (5-bromotryptammonium chloride or 5-Br-T·HCl);
2-(5-chloro-1H-indol-3-yl)ethylazanium chloride (5-chlorotryptammonium chloride or 5-Cl-T·HCl);
2-(5-fluoro-1H-indol-3-yl)ethan-1-aminium chloride (5-fluorotryptammonium chloride or 5-F-T·HCl);
2-(5-methyl-1H-indol-3-yl)ethylazanium chloride (5-methyltryptammonium chloride or 5-Me-T·HCl);
2-(6-fluoro-1H-indol-3-yl)ethan-1-aminium chloride (6-fluorotryptammonium chloride or 6-F-T·HCl); and
2-(7-methyl-1H-indol-3-yl)ethyl]azanium chloride (7-methyltryptammonium chloride or 7-Me-T·HCl).

In one embodiment, this disclosure relates to 1-methyltryptammonium chloride (1-Me-T·HCl), crystalline 1-Me-T·HCl, and specific crystalline forms thereof. 1-Me-T·HCl has the following chemical formula:

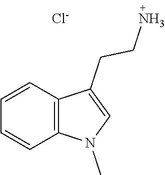

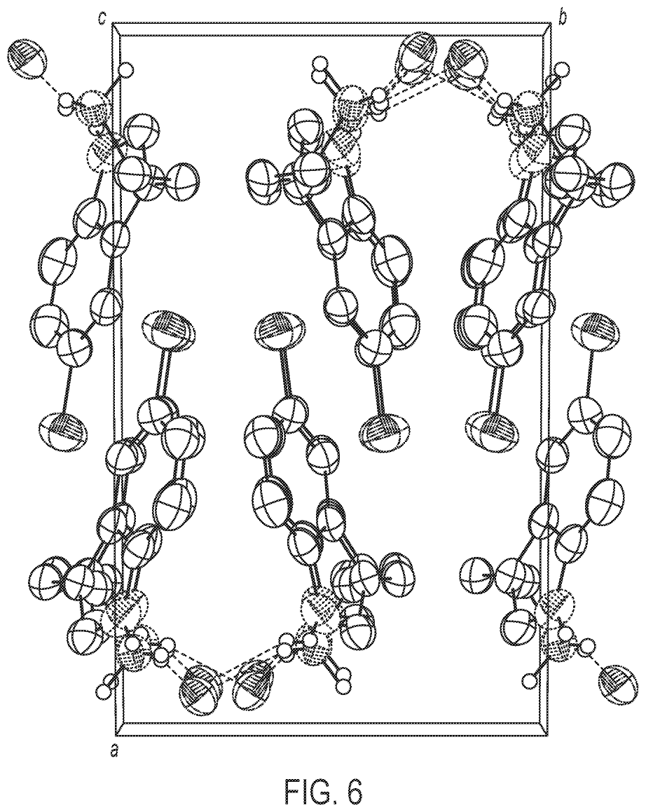

In one embodiment, this disclosure pertains to particular crystalline forms of 1-Me-T·HCl, including crystalline form 1 of 1-Me-T·HCl. In one embodiment, crystalline form 1 of 1-Me-T·HCl is characterized by at least one of: a trigonal, R3c space group at a temperature of about 297(2) K; unit cell dimensions a=29.3337(13), b=29.3337(13), c=7.3922(6), α=900, β=900, and γ=120°; an x-ray powder diffraction (XRPD) pattern substantially similar to FIG. 6; and an XRPD pattern characterized by at least two peaks selected from 6.0, 10.4, 15.1, 16.0, and 19.4 ° 2θ±0.2 ° 2θ.

In one embodiment, this disclosure also relates to 1-phenyl-2-methyltryptammonium chloride (1-Ph-2-Me-T·HCl), crystalline 1-Ph-2-Me-T·HCl, and specific crystalline forms thereof. 1-Ph-2-Me-T·HCl has the following chemical formula:

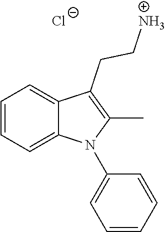 [SMILES: [Cl-] ; Cc1c(CC[NH3+])c2ccccc2n1-c1ccccc1]

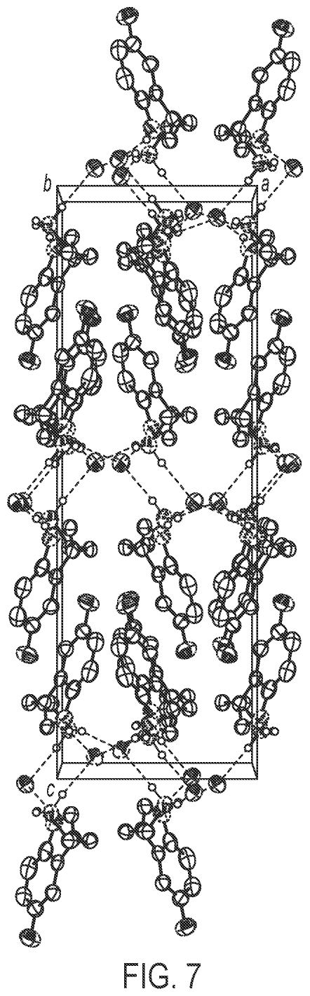

In one embodiment, this disclosure pertains to particular crystalline forms of 1-Ph-2-Me-T·HCl, including crystalline form 1 of 1-Ph-2-Me-T·HCl. In one embodiment, crystalline form 1 of 1-Ph-2-Me-T·HCl is characterized by at least one of: a monoclinic, P2_{1/n} space group at a temperature of about 297(2) K; unit cell dimensions a=10.3990(6), b=16.3016(10), c=37.091(2), α=900, β=97.963(2°), and γ=90°; an x-ray powder diffraction (XRPD) pattern substantially similar to FIG. 7; and an XRPD pattern characterized by at least two peaks selected from 9.0, 15.4, and 17.2 ° 2θ±0.2 ° 2θ.

In one embodiment, this disclosure also relates to 5-methoxytryptammonium chloride (5-MeO-T·HCl), crystalline 5-MeO-T·HCl, and specific crystalline forms thereof. 5-MeO-T·HCl has the following chemical formula:

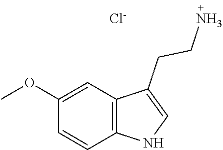

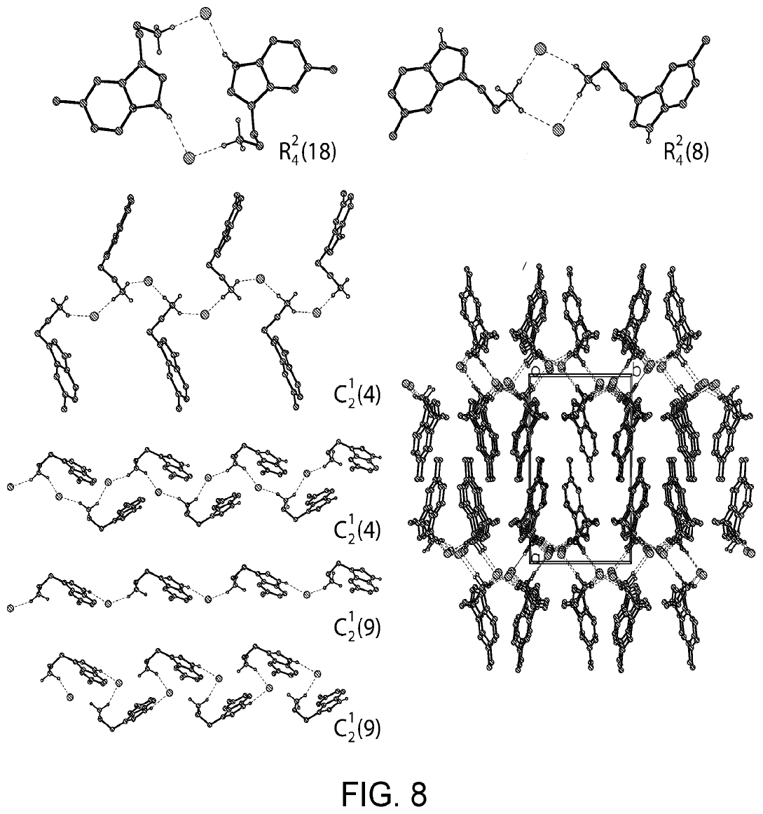

In one embodiment, this disclosure pertains to particular crystalline forms of 5-MeO-T·HCl, including crystalline form 1 of 5-MeO-T·HCl. In one embodiment, crystalline form 1 of 5-MeO-T·HCl is characterized by at least one of: a monoclinic, P2_{1/C} space group at a temperature of about 297(2) K; unit cell dimensions a=14.6858(8), b=8.3613(4), c=9.7878(5), α=900, β=102.742(2°), and γ=90°; an x-ray powder diffraction (XRPD) pattern substantially similar to FIG. 8; and an XRPD pattern characterized by at least two peaks selected from 14.1, 16.2, and 22.6 ° 2θ±0.2 ° 2θ.

In one embodiment, this disclosure also relates to 5-bromotryptammonium chloride (5-Br-T·HCl), crystalline 5-Br-T·HCl, and specific crystalline forms thereof. 5-Br-T·HCl has the following chemical formula:

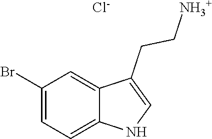 [SMILES: [Cl-] ; [NH3+]CCc1c[nH]c2ccc(Br)cc12]

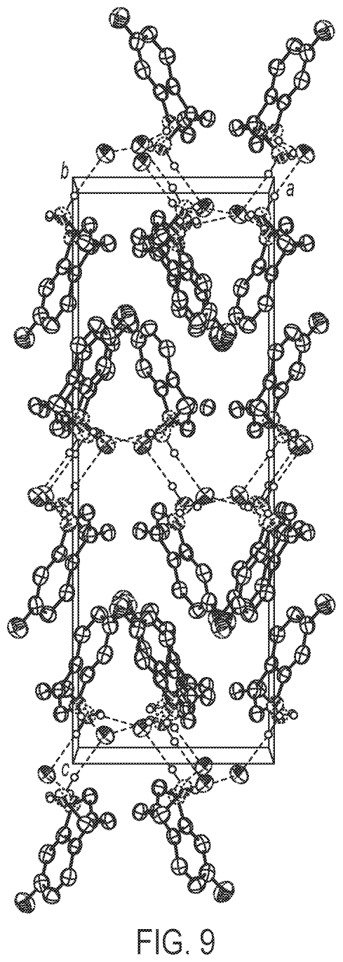

In one embodiment, this disclosure pertains to particular crystalline forms of 5-Br-T·HCl, including crystalline form 1 of 5-Br-T·HCl. In one embodiment, crystalline form 1 of 5-Br-T·HCl is characterized by at least one of: an orthorhombic, Pbca space group at a temperature of about 297(2) K; unit cell dimensions a=8.6153(6), b=9.3766(5), c=29.173(2), α=900, β=90°, and γ=90°; an x-ray powder diffraction (XRPD) pattern substantially similar to FIG. 9; and an XRPD pattern characterized by at least two peaks selected from 6.1, 16.7, and 19.9 ° 2θ±0.2 ° 2θ.

In one embodiment, this disclosure also relates to 5-chlorotryptammonium chloride (5-Cl-T·HCl), crystalline 5-Cl-T·HCl, and specific crystalline forms thereof. 5-Cl-T·HCl has the following chemical formula:

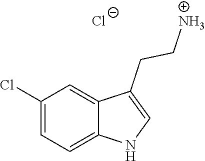 [SMILES: [Cl-] ; [NH3+]CCc1c[nH]c2ccc(Cl)cc12]

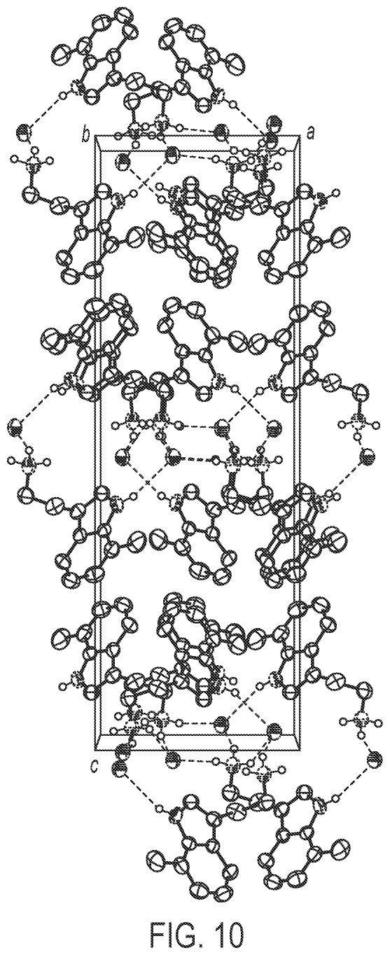

In one embodiment, this disclosure pertains to particular crystalline forms of 5-Cl-T·HCl, including crystalline form 1 of 5-Cl-T·HCl. In one embodiment, crystalline form 1 of 5-Cl-T·HCl is characterized by at least one of: a monoclinic, P2_{1/C} space group at a temperature of about 297(2) K; unit cell dimensions a=14.7030(9), b=8.6058(5), c=9.4141(5), α=900, β=106.450(2°), and γ=90°; an x-ray powder diffraction (XRPD) pattern substantially similar to FIG. 10; and an XRPD pattern characterized by at least two peaks selected from 16.6, 20.1, and 23.0 ° 2θ±0.2 ° 2θ.

In one embodiment, this disclosure also relates to 5-fluorotryptammonium chloride (5-F-T·HCl), crystalline 5-F-T·HCl, and specific crystalline forms thereof. 5-F-T·HCl has the following chemical formula:

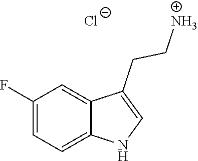 [SMILES: [Cl-] ; [NH3+]CCc1c[nH]c2ccc(F)cc12]

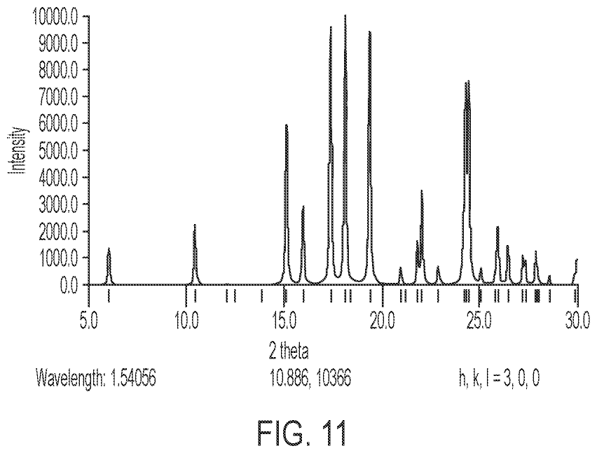

In one embodiment, this disclosure pertains to particular crystalline forms of 5-F-T·HCl, including crystalline form 1 of 5-F-T·HCl. In one embodiment, crystalline form 1 of 5-F-T·HCl is characterized by at least one of: an orthorhombic, Pbca space group at a temperature of about 297(2) K; unit cell dimensions a=8.6708(4), b=9.6684(5), c=25.6854(12), α=900, β=90°, and γ=90°; an x-ray powder diffraction (XRPD) pattern substantially similar to FIG. 11; and an XRPD pattern characterized by at least two peaks selected from 14.1, 15.3, and 17.2 ° 2θ±0.2 ° 2θ.

In one embodiment, this disclosure also relates to 5-methyltryptammonium chloride (5-Me-T·HCl), crystalline 5-Me-T·HCl, and specific crystalline forms thereof. 5-Me-T·HCl has the following chemical formula:

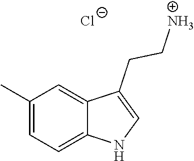

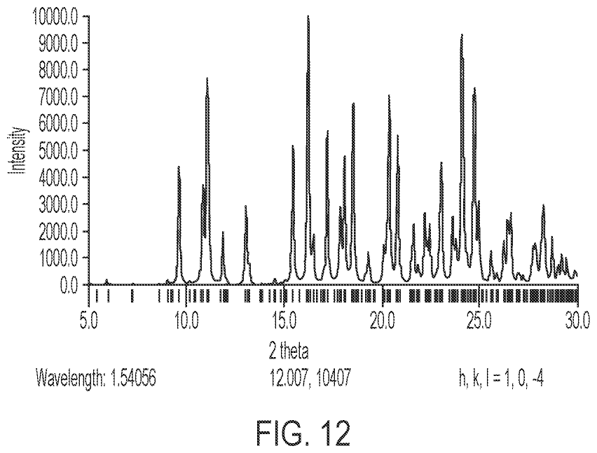

In one embodiment, this disclosure pertains to particular crystalline forms of 5-Me-T·HCl, including crystalline form 1 of 5-Me-T·HCl. In one embodiment, crystalline form 1 of 5-Me-T·HCl is characterized by at least one of: a monoclinic, P2_{1/C} space group at a temperature of about 297(2) K; unit cell dimensions a=14.9939(10), b=8.4270(5), c=9.5388(6), a=90°, β=107.774(2°), and γ=90°; an x-ray powder diffraction (XRPD) pattern substantially similar to FIG. 12; and an XRPD pattern characterized by at least two peaks selected from 6.2, 19.7, and 23.3 ° 2θ±0.2 ° 2θ.

In one embodiment, this disclosure also relates to 6-fluorotryptammonium chloride (6-F-T·HCl), crystalline 6-F-T·HCl, and specific crystalline forms thereof. 6-F-T·HCl has the following chemical formula:

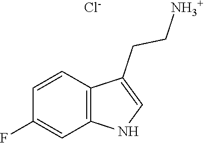 [SMILES: [Cl-] ; [NH3+]CCc1c[nH]c2cc(F)ccc12]

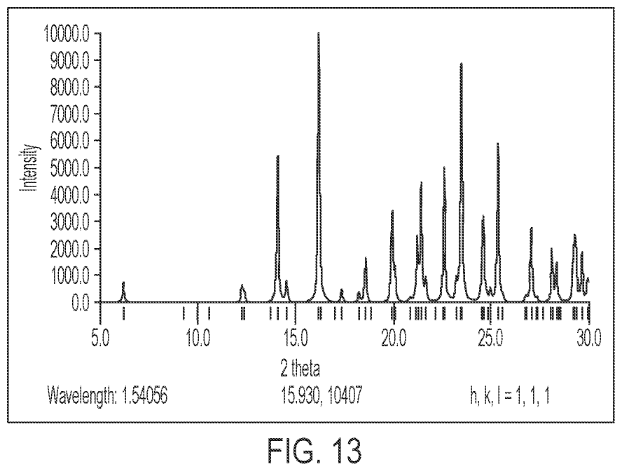

In one embodiment, this disclosure pertains to particular crystalline forms of 6-F-T·HCl, including crystalline form 1 of 6-F-T·HCl. In one embodiment, crystalline form 1 of 6-F-T·HCl is characterized by at least one of: an orthorhombic, Pbca space group at a temperature of about 297(2) K; unit cell dimensions a=8.3572(4), b=10.3493(5), c=24.3824(13), a=90°, β=90°, and γ=90°; an x-ray powder diffraction (XRPD) pattern substantially similar to FIG. 13; and an XRPD pattern characterized by at least two peaks selected from 14.1, 15.4, and 21.9 ° 2θ±0.2 ° 2θ.

In one embodiment, this disclosure also relates to 7-methyltryptammonium chloride (7-Me-T·HCl), crystalline 7-Me-T·HCl, and specific crystalline forms thereof. 7-Me-T·HCl has the following chemical formula:

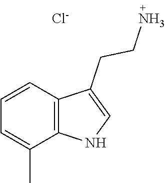

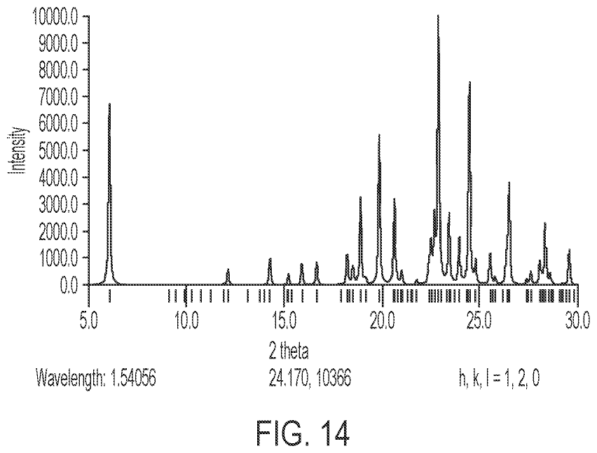

In one embodiment, this disclosure pertains to particular crystalline forms of 7-Me-T·HCl, including crystalline form 1 of 7-Me-T·HCl. In one embodiment, crystalline form 1 of 7-Me-T·HCl is characterized by at least one of: an orthorhombic, Pbca space group at a temperature of about 297(2) K; unit cell dimensions a=9.1893(5), b=9.3259(4), c=27.5149(15), a=90°, β=900, and γ=90°; an x-ray powder diffraction (XRPD) pattern substantially similar to FIG. 14; and an XRPD pattern characterized by at least two peaks selected from 6.4, 15.0, and 18.7 ° 2θ±0.2 ° 2θ.

The disclosure also relates to methods, such as those described in the examples, used to characterize the tryptammonium compounds of the disclosure, crystalline tryptammonium compounds of the disclosure, and specific crystalline forms thereof, such as crystalline form 1 of a tryptammonium compound of the disclosure.

### Methods of Treatment and Therapeutic Uses

The tryptammonium compounds of the disclosure, crystalline tryptammonium compounds of the disclosure, or specific crystalline forms thereof, such as crystalline form 1 of a tryptammonium compound of the disclosure, and the methods and the compositions (e.g., pharmaceutical compositions) are used to regulate the activity of a neurotransmitter receptor by administering a therapeutically effective dose of a tryptammonium compound of the disclosure, a crystalline tryptammonium compound of the disclosure, or a specific crystalline form thereof, such as crystalline form 1 of a tryptammonium compound of the disclosure. In one embodiment, tryptammonium compounds of the disclosure, crystalline tryptammonium compounds of the disclosure, or specific crystalline forms thereof, such as crystalline form 1 of a tryptammonium compound of the disclosure, and the methods and the compositions (e.g., pharmaceutical compositions) are used to treat inflammation and/or pain by administering a therapeutically effective dose of a tryptammonium compound of the disclosure, a crystalline tryptammonium compound of the disclosure, or a specific crystalline form thereof, such as crystalline form 1 of a tryptammonium compound of the disclosure.

Methods of the disclosure also related to the administration of a therapeutically effective amount of a tryptammonium compound of the disclosure, a crystalline tryptammonium compound of the disclosure, or a specific crystalline form thereof, such as crystalline form 1 of a tryptammonium compound of the disclosure, to prevent or treat a disease or condition, such as those discussed below for a subject in need thereof of treatment.

As used herein, the term “a subject in need thereof” refers to a person requiring a composition to treat a particular disease or condition (e.g., inflammation, pain, a psychological disorder, modulating activity at a receptor, etc.). In one embodiment, the “subject in need thereof” may be identified by analyzing, diagnosing, and/or determining whether the person (or subject) requires the composition for treatment of a particular disease or condition. In one embodiment, identifying a person in need of treatment comprises diagnosing a person with a medical condition, e.g., a neurological disorder, a chemical imbalance, a hereditary condition, etc. In one embodiment, identifying a person in need of treatment comprises performing a psychiatric evaluation. In one embodiment, identifying a person in need of treatment comprises performing a blood test. In one embodiment, identifying a person in need of treatment comprises determining whether a person has a compulsive disorder. In one embodiment, identifying a person in need of treatment comprises self-identifying as having a compulsive disorder.

Tryptammonium compounds of the disclosure, crystalline tryptammonium compounds of the disclosure, or specific crystalline forms thereof, such as crystalline form 1 of a tryptammonium compound of the disclosure, may be administered neat or as a composition comprising a tryptammonium compound of the disclosure, a crystalline tryptammonium compound of the disclosure, or a specific crystalline form thereof, such as crystalline form 1 of a tryptammonium compound of the disclosure, as discussed below.

Tryptammonium compounds of the disclosure, crystalline tryptammonium compounds of the disclosure, or specific crystalline forms thereof, such as crystalline form 1 of a tryptammonium compound of the disclosure, may be used to prevent and/or treat a psychological disorder. The disclosure provides a method for preventing and/or treating a psychological disorder by administering to a subject in need thereof a therapeutically effective amount of a tryptammonium compound of the disclosure, a crystalline tryptammonium compounds of the disclosure, or specific crystalline forms thereof, or as crystalline form 1 of a tryptammonium compound of the disclosure, including the exemplary embodiments discussed herein. The psychological disorder may be chosen from depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome, post-traumatic stress disorder (PTSD), premenstrual dysphoric disorder (PMDD), and premenstrual syndrome (PMS).

Tryptammonium compounds of the disclosure, crystalline tryptammonium compounds of the disclosure, or specific crystalline forms thereof, such as crystalline form 1 of a tryptammonium compound of the disclosure, may be used to prevent and/or treat a brain disorder. The disclosure provides a method for preventing and/or treating a brain disorder (e.g., Huntington's disease, Alzheimer's disease, dementia, and Parkinson's disease) by administering to a subject in need thereof a therapeutically effective amount of a tryptammonium compound of the disclosure, a crystalline tryptammonium compound of the disclosure, or a specific crystalline form thereof, such as crystalline form 1 of a tryptammonium compound of the disclosure.

Tryptammonium compounds of the disclosure, crystalline tryptammonium compounds of the disclosure, or specific crystalline forms thereof, such as crystalline form 1 of a tryptammonium compound of the disclosure, may be used to prevent and/or treat developmental disorders, delirium, dementia, amnestic disorders and other cognitive disorders, psychiatric disorders due to a somatic condition, drug-related disorders, schizophrenia and other psychotic disorders, mood disorders, anxiety disorders, somatoform disorders, factitious disorders, dissociative disorders, eating disorders, sleep disorders, impulse control disorders, adjustment disorders, or personality disorders. The disclosure provides a method for preventing and/or treating these disorders by administering to a subject in need thereof a therapeutically effective amount of a tryptammonium compound of the disclosure, a crystalline tryptammonium compounds of the disclosure, or a specific crystalline form thereof, such as crystalline form 1 of a tryptammonium compound of the disclosure.

Tryptammonium compounds of the disclosure, crystalline tryptammonium compounds of the disclosure, or specific crystalline forms thereof, such as crystalline form 1 of a tryptammonium compound of the disclosure, may be used to prevent and/or treat inflammation and/or pain, such as for example inflammation and/or pain associated with inflammatory skeletal or muscular diseases or conditions. The disclosure provides a method for preventing and/or treating an inflammation and/or pain by administering to a subject in need thereof a therapeutically effective amount of a tryptammonium compound of the disclosure, a crystalline tryptammonium compound of the disclosure, or a specific crystalline form thereof, such as crystalline form 1 of a tryptammonium compound of the disclosure, including the exemplary embodiments discussed herein. Generally speaking, treatable “pain” includes nociceptive, neuropathic, and mix-type. A method of the disclosure may reduce or alleviate the symptoms associated with inflammation, including but not limited to treating localized manifestation of inflammation characterized by acute or chronic swelling, pain, redness, increased temperature, or loss of function in some cases. A method of the disclosure may reduce or alleviate the symptoms of pain regardless of the cause of the pain, including but not limited to reducing pain of varying severity, i.e., mild, moderate and severe pain, acute pain and chronic pain. A method of the disclosure is effective in treating joint pain, muscle pain, tendon pain, burn pain, and pain caused by inflammation such as rheumatoid arthritis. Skeletal or muscular diseases or conditions which may be treated include but are not limited to musculoskeletal sprains, musculoskeletal strains, tendinopathy, peripheral radiculopathy, osteoarthritis, joint degenerative disease, polymyalgia rheumatica, juvenile arthritis, gout, ankylosing spondylitis, psoriatic arthritis, systemic lupus erythematosus, costochondritis, tendonitis, bursitis, such as the common lateral epicondylitis (tennis elbow), medial epicondylitis (pitchers elbow) and trochanteric bursitis, temporomandibularjoint syndrome, and fibromyalgia.

Tryptammonium compounds of the disclosure, crystalline tryptammonium compounds of the disclosure, or specific crystalline forms thereof, such as crystalline form 1 of a tryptammonium compound of the disclosure, may be used to modulate activity of a mitogen activating protein (MAP), comprising administering a composition of the disclosure. In one embodiment, the mitogen activating protein (MAP) comprises a MAP kinase (MAPk). MAPKs provide a wide-ranging signaling cascade that allow cells to quickly respond to biotic and abiotic stimuli. Exemplary MAPKs include, but are not limited to, Tropomyosin Receptor Kinase A (TrkA), P38-alpha, Janus Kinase 1 (JAK1), and c-Jun N-Terminal Kinase 3 (JNK3). TrkA is a high affinity catalytic receptor of nerve growth factor (NGF) protein. TrkA regulates NGF response, influencing neuronal differentiation and outgrowth as well as programmed cell death. p38-alpha is involved with the regulation of pro-inflammatory cytokines, including TNF-α. In the central nervous system, p38-alpha regulates neuronal death and neurite degeneration, and it is a common target of Alzheimer's disease therapies. JAK1 influences cytokine signaling, including IL-2, IL-4, IFN-alpha/beta, IFN-γ, and IL-10, and it is implicated in brain aging. JNK3 is neuronal specific protein isoform of the JNKs. It is involved with the regulation of apoptosis. JNK3 also plays a role in modulating the response of cytokines, growth factors, and oxidative stress.

As used herein, the term “modulating activity of a mitogen activating protein” refers to changing, manipulating, and/or adjusting the activity of a mitogen activating protein. In one embodiment, modulating the activity of a MAP, such as a MAPK, can influence neural health, neurogenesis, neural growth and differentiation, and neurodegenerative diseases.

Tryptammonium compounds of the disclosure, crystalline tryptammonium compounds of the disclosure, or specific crystalline forms thereof, such as crystalline form 1 of a tryptammonium compound of the disclosure, may be used to modulate neurogenesis, comprising administering a composition of the disclosure. As used herein, the term “modulating neurogenesis” refers to changing, manipulating, and/or adjusting the growth and development of neural tissue. In one embodiment, neurogenesis comprises adult neurogenesis, in which new neural stem cells are generated from neural stem cells in an adult animal. In one embodiment, modulating neurogenesis comprises increasing and/or enhancing the rate at which new neural tissue is developed.

Tryptammonium compounds of the disclosure, crystalline tryptammonium compounds of the disclosure, or specific crystalline forms thereof, such as crystalline form 1 of a tryptammonium compound of the disclosure, may be used to modulate neurite outgrowth, comprising administering a composition of the disclosure. As used herein, the term “modulating neurite outgrowth” refers to changing, manipulating, and/or adjusting the growth and development of neural projections, or “neurites.” In one embodiment, neurogenesis comprises modulating the growth of new neurites, the number of neurites per neuron, and/or neurite length. In one embodiment, modulating neurite outgrowth comprises increasing and/or enhancing the rate and/or length at which neurites develop.

This disclosure also relates to methods of preventing or treating sexual health disorders including, but not limited to, hypoactive sexual desire disorder, hyperactive sexual desire disorder, orgasmic disorder, arousal disorder, vaginismus, and dyspareunia. In some embodiments, the disorder is a male sexual dysfunction disorder. In some embodiments, the disorder is a female sexual dysfunction disorder.

This disclosure also relates to methods of preventing or treating women's health disorders including, but not limited to, menstrual cramping, dysmenorrhea, post-hysterectomic pain, vaginal or vulvar vestibule mucosa disorder, menopausal-related disorders, vaginal atrophy, or vulvar vestibulitis.

### Compositions

The disclosure also relates to compositions comprising an effective amount of a tryptammonium compound of the disclosure, a crystalline tryptammonium compound of the disclosure, or a specific crystalline form thereof, such as crystalline form 1 of a tryptammonium compound of the disclosure, and an excipient (e.g., a pharmaceutically-acceptable excipient). In another embodiment, the disclosure also relates to pharmaceutical compositions comprising a therapeutically effective amount of a tryptammonium compound of the disclosure, a crystalline tryptammonium compound of the disclosure, or a specific crystalline form thereof, such as crystalline form 1 of a tryptammonium compound of the disclosure, and a pharmaceutically acceptable excipient (also known as a pharmaceutically acceptable carrier). As discussed above, a tryptammonium compound of the disclosure, a crystalline tryptammonium compound of the disclosure, or a specific crystalline form thereof, such as crystalline form 1 of a tryptammonium compound of the disclosure, may be, for example, therapeutically useful to prevent and/or treat the psychological disorders, brain disorders, pain, and inflammation as well as the other disorders described herein.

A composition or a pharmaceutical composition of the disclosure may be in any form which contains a tryptammonium compound of the disclosure, a crystalline tryptammonium compound of the disclosure, or a specific crystalline form thereof, such as crystalline form 1 of a tryptammonium compound of the disclosure. The composition may be, for example, a tablet, capsule, liquid suspension, injectable, topical, or transdermal. The compositions generally contain, for example, about 1% to about 99% by weight of a tryptammonium compound of the disclosure, a crystalline tryptammonium compound of the disclosure, or a specific crystalline form thereof, such as crystalline form 1 of a tryptammonium compound of the disclosure, and, for example, 99% to 1% by weight of at least one suitable pharmaceutically acceptable excipient. In one embodiment, the composition may be between about 5% and about 75% by weight of a tryptammonium compound of the disclosure, a crystalline tryptammonium compound of the disclosure, or a specific crystalline form thereof, such as crystalline form 1 of a tryptammonium compound of the disclosure, with the rest being at least one suitable pharmaceutically acceptable excipient or at least one other adjuvant, as discussed below.

Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. Various ratios of these components in the composition are also disclosed. The disclosures of US 2018/0221396 A1 and US 2019/0142851 A1 are incorporated herein by reference. According to this disclosure, a tryptammonium compound of the disclosure, a crystalline tryptammonium compound of the disclosure, or a specific crystalline form thereof, such as crystalline form 1 of a tryptammonium compound of the disclosure, may be used as the “first purified psilocybin derivative” in the compositions described in US 2018/0221396 A1 and US 2019/0142851 A1. Accordingly, this disclosure provides a composition comprising: a first component comprising at least one tryptammonium compound of the disclosure, crystalline tryptammonium compound of the disclosure, or specific crystalline form thereof, such as crystalline form 1 of a tryptammonium compound of the disclosure; at least one second component selected from at least one of (a) a serotonergic drug, (b) a purified psilocybin derivative, (c) a purified cannabinoid or (d) a purified terpene; and at least one pharmaceutically-acceptable excipient or at least one other adjuvant. Such a composition may be a pharmaceutical composition wherein the components are present individually in therapeutically effective amounts or by combination in a therapeutically effective amount to treat a disease, disorder, or condition as described herein.

When used in such compositions as a first component comprising at least one tryptammonium compound of the disclosure, crystalline tryptammonium compound of the disclosure, or specific crystalline form thereof, such as crystalline form 1 of a tryptammonium compound of the disclosure, with a second component selected from at least one of (a) a serotonergic drug, (b) a purified psilocybin derivative, (c) a purified cannabinoid, or (d) a purified terpene, the compositions represent particular embodiments of the disclosure. Compositions having as a first component at least one tryptammonium compound of the disclosure, crystalline tryptammonium compound of the disclosure, or specific crystalline form thereof, such as crystalline form 1 of a tryptammonium compound of the disclosure with a second component selected from at least one of (e) an adrenergic drug, (f) a dopaminergic drug, (g) a monoamine oxidase inhibitor, (h) a purified erinacine, (i) a purified hericenone represent additional particular embodiments of the disclosure represented by the compositions having tryptammonium compounds of the disclosure, crystalline tryptammonium compounds of the disclosure, or specific crystalline forms thereof, such as crystalline form 1 of a tryptammonium compound of the disclosure. In some embodiments, the first and second components can be administered at the same time (e.g., together in the same composition), or at separate times over the course of treating a patient in need thereof. Such a composition may be a pharmaceutical composition wherein the components are present individually in therapeutically effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments. Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed exemplary embodiments. Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments. Exemplary terpenes include but are not limited to the terpenes described in paragraphs [0160]-[0238] of US 2018/0221396 A1 and [0161]-[0300] US 2019/0142851 A1 as well as the disclosed exemplary embodiments.

A pharmaceutical formulation of the disclosure may comprise, consist essentially of, or consist of (a) at least one tryptammonium compound of the disclosure, crystalline tryptammonium compound of the disclosure, or specific crystalline form thereof, such as crystalline form 1 of a tryptammonium compound of the disclosure, and (b) at least one second active compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and (c) a pharmaceutically acceptable excipient. In some embodiments, the tryptammonium compounds of the disclosure, crystalline tryptammonium compounds of the disclosure, or specific crystalline forms thereof, such as crystalline form 1 of a tryptammonium compound of the disclosure, and the second active compound(s) are each present in a therapeutically effective amount using a purposefully engineered and unnaturally occurring molar ratios. Exemplary molar ratios of the tryptammonium compounds of the disclosure, crystalline tryptammonium compounds of the disclosure, or specific crystalline forms thereof, such as crystalline form 1 of a tryptammonium compound of the disclosure, to the second active compound in a composition of the disclosure include but are not limited to from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

A pharmaceutical formulation of the disclosure may comprise a composition containing a tryptammonium compound of the disclosure, a crystalline tryptammonium compound of the disclosure, or a specific crystalline form thereof, such as crystalline form 1 of a tryptammonium compound of the disclosure, and a serotonergic drug, a purified psilocybin derivative, a purified cannabinoid, or a purified terpene, each present in a therapeutically effective amount using a purposefully engineered and unnaturally occurring molar ratios. Published US applications US 2018/0221396 A1 and US 2019/0142851 A1 disclose compositions comprising a combination of a purified psilocybin derivative with a second purified psilocybin derivative, with one or two purified cannabinoids or with a purified terpene. According to this disclosure composition containing a tryptammonium compound of the disclosure, a crystalline tryptammonium compound of the disclosure, or a specific crystalline form thereof, such as crystalline form 1 of a tryptammonium compound of the disclosure, may be used in place of a “purified psilocybin derivative” in the compositions described in US 2018/0221396 A1 and US 2019/0142851 A1. Accordingly, the disclosure provides a pharmaceutical formulation comprising as (a) at least one tryptammonium compound of the disclosure, crystalline tryptammonium compound of the disclosure, or specific crystalline form thereof, such as crystalline form 1 of a tryptammonium compound of the disclosure, and at least one second component selected from (b) a purified psilocybin derivative, (c) a purified cannabinoid or (d) a purified terpene; and at least one pharmaceutically-acceptable excipient or at least one other adjuvant, as described herein. Such a composition may be a pharmaceutical composition wherein the components are present individually in therapeutic effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments. Some exemplary serotonergic drugs include SSRIs and SNRIs. Some examples of specific serotonergic drugs include the following molecules, including any salts, solvates, or polymorphs thereof: 6-Allyl-N,N-diethyl-NL, N,N-Dibutyl-T, N,N-Diethyl-T, N,N-Diisopropyl-T, 5-Methyoxy-alpha-methyl-T, N,N-Dimethyl-T, 2,alpha-Dimethyl-T, alpha,N-Dimethyl-T, N,N-Dipropyl-T, N-Ethyl-N-isopropyl-T, alpha-Ethyl-T, 6,N,N-Triethyl-NL, 3,4-Dihydro-7-methoxy-1-methyl-C, 7-Methyoxy-1-methyl-C, N,N-Dibutyl-4-hydroxy-T, N,N-Diethyl-4-hydroxy-T, N,N-Diisopropyl-4-hydroxy-T, N,N-Dimethyl-4-hydroxy-T, N,N-Dimethyl-5-hydroxy-T, N, N-Dipropyl-4-hydroxy-T, N-Ethyl-4-hydroxy-N-methyl-T, 4-Hydroxy-N-isopropyl-N-methyl-T, 4-Hydroxy-N-methyl-N-propyl-T, 4-Hydroxy-N,N-tetramethylene-T Ibogaine, N,N-Diethyl-L, N-Butyl-N-methyl-T, N,N-Diisopropyl-4,5-methylenedioxy-T, N,N-Diisopropyl-5,6-methylenedioxy-T, N,N-Dimethyl-4,5-methylenedioxy-T, N,N-Dimethyl-5,6-methylenedioxy-T, N-Isopropyl-N-methyl-5,6-methylenedioxy-T, N,N-Diethyl-2-methyl-T, 2,N,N-Trimethyl-T, N-Acetyl-5-methoxy-T, N,N-Diethyl-5-methoxy-T, N,N-Diisopropyl-5-methoxy-T, 5-Methoxy-N,N-dimethyl-T, N-Isopropyl-4-methoxy-N-methyl-T, N-Isopropyl-5-methoxy-N-methyl-T, 5,6-Dimethoxy-N-isopropyl-N-methyl-T, 5-Methoxy-N-methyl-T, 5-Methoxy-N,N-tetramethylene-T, 6-Methoxy-1-methyl-1,2,3,4-tetrahydro-C, 5-Methoxy-2,N,N-trimethyl-T, N,N-Dimethyl-5-methylthio-T, N-Isopropyl-N-methyl-T, alpha-Methyl-T, N-Ethyl-T, N-Methyl-T, 6-Propyl-N L, N,N-Tetramethylene-T, Tryptamine, and 7-Methoxy-1-methyl-1,2,3,4-tetrahydro-C, alpha,N-Dimethyl-5-methoxy-T. For additional information regarding these compounds see Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof. In an exemplary embodiment, the serotonergic drug is 3,4-methylenedioxymethamphetamine.

Exemplary psilocybin derivatives include but are not limited to psilocybin itself and the psilocybin derivates described in paragraphs [0081]-[0109] of US 2018/0221396 A1 and [082]-[0110] US 2019/0142851 A1 as well as the disclosed exemplary embodiments. In one embodiment, the compositions disclosed herein comprise one or more purified psilocybin derivatives chosen from: [3-(2-Dimethylaminoethyl)-1H-indol-4-yl] dihydrogen phosphate, 4-hydroxytryptamine, 4-hydroxy-N,N-dimethyltryptamine, [3-(2-methylaminoethyl)-1H-indol-4-yl] dihydrogen phosphate, 4-hydroxy-N-methyltryptamine, [3-(aminoethyl)-1H-indol-4-yl] dihydrogen phosphate, [3-(2-trimethylaminoethyl)-1H-indol-4-yl] dihydrogen phosphate, and 4-hydroxy-N,N,N-trimethyltryptamine.

Exemplary cannabinoids include but are not limited to the cannabinoids described in paragraphs [0111]-[0159] of US 2018/0221396 A1 and [0112]-[0160] US 2019/0142851 A1 as well as the disclosed exemplary embodiments, incorporated here by reference. Examples of cannabinoids within the context of this disclosure include the following molecules: Cannabichromene (CBC), Cannabichromenic acid (CBCA), Cannabichromevarin (CBCV), Cannabichromevarinic acid (CBCVA), Cannabicyclol (CBL), Cannabicyclolic acid (CBLA), Cannabicyclovarin (CBLV), Cannabidiol (CBD), Cannabidiol monomethylether (CBDM), Cannabidiolic acid (CBDA), Cannabidiorcol (CBD-C1), Cannabidivarin (CBDV), Cannabidivarinic acid (CBDVA), Cannabielsoic acid B (CBEA-B), Cannabielsoin (CBE), Cannabielsoin acid A (CBEA-A), Cannabigerol (CBG), Cannabigerol monomethylether (CBGM), Cannabigerolic acid (CBGA), Cannabigerolic acid monomethylether (CBGAM), Cannabigerovarin (CBGV), Cannabigerovarinic acid (CBGVA), Cannabinodiol (CBND), Cannabinodivarin (CBDV), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1), Cannabivarin (CBV), Cannabitriol (CBT), Cannabitriolvarin (CBTV), 10-Ethoxy-9-hydroxy-delta-6a-tetrahydrocannabinol, Cannbicitran (CBT), Cannabiripsol (CBR), 8,9-Dihydroxy-delta-6a-tetrahydrocannabinol, Delta-8-tetrahydrocannabinol (A8-THC), Delta-8-tetrahydrocannabinolic acid (A8-THCA), Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV), Delta-9-tetrahydrocannabivarinic acid (THCVA), 10-Oxo-delta-6a-tetrahydrocannabinol (OTHC), Cannabichromanon (CBCF), Cannabifuran (CBF), Cannabiglendol, Delta-9-cis-tetrahydrocannabinol (cis-THC), Tryhydroxy-delta-9-tetrahydrocannabinol (triOH-THC), Dehydrocannabifuran (DCBF), and 3,4,5,6-Tetrahydro-7-hydroxy-alpha-alpha-2-trimethyl-9-n-propyl-2,6-metha-no-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed exemplary embodiments. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, caryophyllene, caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, caryophyllene, cedrene, cymene, elemene, eucalyptol, eudesmol, farnesene, fenchol, geraniol, guaiacol, humulene, isoborneol, limonene, linalool, menthol, myrcene, nerolidol, ocimene, phellandrene, phytol, pinene, pulegone, sabinene, terpineol, terpinolene, or valencene.

As used herein, the term “adrenergic drug” refers to a compound that binds, blocks, or otherwise influences (e.g., via an allosteric reaction) activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.).

In one embodiment, an adrenergic drug is an antidepressant. In one embodiment, an adrenergic drug is a norepinephrine transporter inhibitor. In one embodiment, an adrenergic drug is a vesicular monoamine transporter inhibitor. In one embodiment, an adrenergic drug is chosen from adrenaline, agmatine, amoxapine, aptazapine, atomoxetine, bupropion, clonidine, doxepin, duloxetine, esmirtazpine, mianserin, ketanserin, mirabegron, mirtazapine, norepinephrine, phentolamine, phenylephrine, piperoxan, reserpine, ritodrine, setiptiline, tesofensine, timolol, trazodone, trimipramine, or xylazine.

As used herein, the term “dopaminergic drug” refers to a compound that binds, blocks, or otherwise influences (e.g., via an allosteric reaction) activity at a dopamine receptor. In one embodiment, a dopaminergic drug binds to a dopamine receptor. In one embodiment, a dopaminergic drug indirectly affects a dopamine receptor, e.g., via interactions affecting the reactivity of other molecules at the dopamine receptor. In one embodiment, a dopaminergic drug is an agonist, e.g., a compound activating a dopamine receptor. In one embodiment, a dopaminergic drug is an antagonist, e.g., a compound binding but not activating a dopamine receptor, e.g., blocking a receptor. In one embodiment, a dopaminergic drug is an effector molecule, e.g., a compound binding to an enzyme for allosteric regulation. In one embodiment, a dopaminergic drug acts (either directly or indirectly) at more than one type of receptor (e.g., 5HT, dopamine, adrenergic, acetylcholine, etc.).

In one embodiment, a dopaminergic drug is a dopamine transporter inhibitor. In one embodiment, a dopaminergic drug is a vesicular monoamine transporter inhibitor. In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, or thioridazine.

As used herein, the term “monoamine oxidase inhibitor” (MAOI) refers to a compound that blocks the actions of monoamine oxidase enzymes. In on embodiment, a MAOI inhibits the activity of one or both monoamine oxidase A and monoamine oxidase B. In one embodiment a MAOI is a reversible inhibitors of monoamine oxidase A. In one embodiment a MAOI is a drug chosen from isocarboxazid, phenelzine, or tranylcypromine. In one embodiment, a MAOI is β-carboline, pinoline, harmane, harmine, harmaline, harmalol, tetrahydroharmine, 9-methyl-ρ-carboline, or 3-carboxy-tetrahydrononharman.

In one embodiment, the compositions and methods disclosed herein include one or more purified erinacine molecules. In one embodiment, the compositions and methods disclosed herein comprise purified erinacine A. In one embodiment, the compositions and methods disclosed herein comprise erinacine B. In one embodiment, the compositions and methods disclosed herein comprise erinacine C. In one embodiment, the compositions and methods disclosed herein comprise erinacine D. In one embodiment, the compositions and methods disclosed herein comprise erinacine E. In one embodiment, the compositions and methods disclosed herein comprise erinacine F. In one embodiment, the compositions and methods disclosed herein comprise erinacine G. In one embodiment, the compositions and methods disclosed herein comprise erinacine H. In one embodiment, the compositions and methods disclosed herein comprise erinacine I. In one embodiment, the compositions and methods disclosed herein comprise erinacine J. In one embodiment, the compositions and methods disclosed herein comprise erinacine K In one embodiment, the compositions and methods disclosed herein comprise erinacine P. In one embodiment, the compositions and methods disclosed herein comprise erinacine Q.

In one embodiment, the compositions and methods disclosed herein comprise erinacine R. In one embodiment, the compositions and methods disclosed herein comprise erinacine S.

In one embodiment, the compositions and methods disclosed herein include one or more purified hericenone molecules. In one embodiment, the compositions and methods disclosed herein comprise purified hericenone A. In one embodiment, the compositions and methods disclosed herein comprise purified hericenone B. In one embodiment, the compositions and methods disclosed herein comprise purified hericenone C. In one embodiment, the compositions and methods disclosed herein comprise purified hericenone D. In one embodiment, the compositions and methods disclosed herein comprise purified hericenone E. In one embodiment, the compositions and methods disclosed herein comprise purified hericenone F. In one embodiment, the compositions and methods disclosed herein comprise purified hericenone G. In one embodiment, the compositions and methods disclosed herein comprise purified hericenone H.

Exemplary compositions of a tryptammonium compound of the disclosure, a crystalline tryptammonium compound of the disclosure, or a specific crystalline form thereof, such as crystalline form 1 of a tryptammonium compound of the disclosure, and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone in exemplary molar ratios are shown in Table 1. A tryptammonium compound of the disclosure, a crystalline tryptammonium compound of the disclosure, or a specific crystalline form thereof, such as crystalline form 1 of a tryptammonium compound of the disclosure, may be any one of the exemplary embodiments described above including their crystalline forms as disclosed herein.

TABLE 1

| | Molar ratio of a | Molar ratio of a | Molar ratio of a |
|---|---|---|---|
| | tryptammonium | tryptammonium | tryptammonium |
| | compound of the | compound of the | compound of the |
| | disclosure, a | disclosure, a | disclosure, a |
| | crystalline | crystalline | crystalline |
| | tryptammonium | tryptammonium | tryptammonium |
| | compound of the | compound of the | compound of the |
| | disclosure, or a | disclosure, or a | disclosure, or a |
| | specific crystalline | specific crystalline | specific crystalline |
| | form thereof, such | form thereof, such | form thereof, such |
| | as crystalline form | as crystalline form | as crystalline form |
| | 1 of a | 1 of a | 1 of a |
| | tryptammonium | tryptammonium | tryptammonium |
| | compound of the | compound of the | compound of the |
| | disclosure:second | disclosure:second | disclosure:second |
| Second Compound | compound | compound | compound |
| 3,4- | About 1:100 to | About 1:25 to | About 1:5 to about |
| methylenedioxymethamphetamine | about 100:1 | about 25:1 | 5:1 |
| Citalopram | About 1:100 to | About 1:25 to | About 1:5 to about |
| | about 100:1 | about 25:1 | 5:1 |
| Escitalopram | About 1:100 to | About 1:25 to | About 1:5 to about |
| | about 100:1 | about 25:1 | 5:1 |
| Fluoxetine | About 1:100 to | About 1:25 to | About 1:5 to about |
| | about 100:1 | about 25:1 | 5:1 |
| Paroxetine | About 1:100 to | About 1:25 to | About 1:5 to about |
| | about 100:1 | about 25:1 | 5:1 |
| Sertraline | About 1:100 to | About 1:25 to | About 1:5 to about |
| | about 100:1 | about 25:1 | 5:1 |
| Duloxetine | About 1:100 to | About 1:25 to | About 1:5 to about |
| | about 100:1 | about 25:1 | 5:1 |
| [3-(2-Dimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxytryptamine | About 1:100 to | About 1:25 to | About 1:5 to about |
| | about 100:1 | about 25:1 | 5:1 |
| 4-hydroxy-N,N-dimethyltryptamine | About 1:100 to | About 1:25 to | About 1:5 to about |
| | about 100:1 | about 25:1 | 5:1 |
| [3-(2-methylaminoethyl)-1H-indol- | About 1:100 to | About 1:25 to | About 1:5 to about |
| 4-yl] dihydrogen phosphate | about 100:1 | about 25:1 | 5:1 |
| 4-hydroxy-N-methyltryptamine | About 1:100 to | About 1:25 to | About 1:5 to about |
| | about 100:1 | about 25:1 | 5:1 |
| [3-(aminoethyl)-1H-indol-4-yl] | About 1:100 to | About 1:25 to | About 1:5 to about |
| dihydrogen phosphate | about 100:1 | about 25:1 | 5:1 |
| [3-(2-trimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxy-N,N,N- | About 1:100 to | About 1:25 to | About 1:5 to about |
| trimethyltryptamine | about 100:1 | about 25:1 | 5:1 |
| THC | About 1:100 to | About 1:25 to | About 1:5 to about |
| | about 100:1 | about 25:1 | 5:1 |
| CBC | About 1:100 to | About 1:25 to | About 1:5 to about |
| | about 100:1 | about 25:1 | 5:1 |
| CBD | About 1:100 to | About 1:25 to | About 1:5 to about |
| | about 100:1 | about 25:1 | 5:1 |
| CBG | About 1:100 to | About 1:25 to | About 1:5 to about |
| | about 100:1 | about 25:1 | 5:1 |
| Myrcene | About 1:100 to | About 1:25 to | About 1:5 to about |
| | about 100:1 | about 25:1 | 5:1 |
| Pinene | About 1:100 to | About 1:25 to | About 1:5 to about |
| | about 100:1 | about 25:1 | 5:1 |
| Caryophyllene | About 1:100 to | About 1:25 to | About 1:5 to about |
| | about 100:1 | about 25:1 | 5:1 |
| Limonene | About 1:100 to | About 1:25 to | About 1:5 to about |
| | about 100:1 | about 25:1 | 5:1 |
| Humulene | About 1:100 to | About 1:25 to | About 1:5 to about |
| | about 100:1 | about 25:1 | 5:1 |
| Linalool | About 1:100 to | About 1:25 to | About 1:5 to about |
| | about 100:1 | about 25:1 | 5:1 |
| Adrenaline | About 1:100 to | About 1:25 to | About 1:5 to about |
| | about 100:1 | about 25:1 | 5:1 |
| Amineptine | About 1:100 to | About 1:25 to | About 1:5 to about |
| | about 100:1 | about 25:1 | 5:1 |
| Erinacine A | About 1:100 to | About 1:25 to | About 1:5 to about |
| | about 100:1 | about 25:1 | 5:1 |
| Hericenone A | About 1:100 to | About 1:25 to | About 1:5 to about |
| | about 100:1 | about 25:1 | 5:1 |
| Phenelzine | About 1:100 to | About 1:25 to | About 1:5 to about |
| | about 100:1 | about 25:1 | 5:1 |

Exemplary pharmaceutical compositions of a tryptammonium compound of the disclosure, a crystalline tryptammonium compound of the disclosure, or a specific crystalline form thereof, such as crystalline form 1 of a tryptammonium compound of the disclosure, and a second compound selected from a serotonergic drug, a purified psilocybin derivative, a purified cannabinoid, a purified terpene, an adrenergic drug, a dopaminergic drug, a monoamine oxidase inhibitor, a purified erinacine, or a purified hericenone and an excipient with exemplary molar ratios of a tryptammonium compound of the disclosure, a crystalline tryptammonium compound of the disclosure, or a specific crystalline form thereof, such as crystalline form 1 of a tryptammonium compound of the disclosure, to the second compound are shown in Table 2. Astryptammonium compound of the disclosure, a crystalline tryptammonium compound of the disclosure, or a specific crystalline form thereof, such as crystalline form 1 of a tryptammonium compound of the disclosure, may be any one of the exemplary embodiments described above including their crystalline forms as disclosed herein.

TABLE 2

| | Molar ratio of a | Molar ratio of a | Molar ratio of a |
|---|---|---|---|
| | tryptammonium | tryptammonium | tryptammonium |
| | compound of the | compound of the | compound of the |
| | disclosure, a | disclosure, a | disclosure, a |
| | crystalline | crystalline | crystalline |
| | tryptammonium | tryptammonium | tryptammonium |
| | compound of the | compound of the | compound of the |
| | disclosure, or a | disclosure, or a | disclosure, or a |
| | specific crystalline | specific crystalline | specific crystalline |
| | form thereof, such | form thereof, such | form thereof, such |
| | as crystalline form | as crystalline form | as crystalline form |
| | 1 of a | 1 of a | 1 of a |
| | tryptammonium | tryptammonium | tryptammonium |
| | compound of the | compound of the | compound of the |
| | disclosure:second | disclosure:second | disclosure:second |
| Second Compound | compound | compound | compound |
| 3,4- | About 1:100 to | About 1:25 to | About 1:5 to about |
| methylenedioxymethamphetamine | about 100:1 | about 25:1 | 5:1 |
| Citalopram | About 1:100 to | About 1:25 to | About 1:5 to about |
| | about 100:1 | about 25:1 | 5:1 |
| Escitalopram | About 1:100 to | About 1:25 to | About 1:5 to about |
| | about 100:1 | about 25:1 | 5:1 |
| Fluoxetine | About 1:100 to | About 1:25 to | About 1:5 to about |
| | about 100:1 | about 25:1 | 5:1 |
| Paroxetine | About 1:100 to | About 1:25 to | About 1:5 to about |
| | about 100:1 | about 25:1 | 5:1 |
| Sertraline | About 1:100 to | About 1:25 to | About 1:5 to about |
| | about 100:1 | about 25:1 | 5:1 |
| Duloxetine | About 1:100 to | About 1:25 to | About 1:5 to about |
| | about 100:1 | about 25:1 | 5:1 |
| [3-(2-Dimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxytryptamine | About 1:100 to | About 1:25 to | About 1:5 to about |
| | about 100:1 | about 25:1 | 5:1 |
| 4-hydroxy-N,N-dimethyltryptamine | About 1:100 to | About 1:25 to | About 1:5 to about |
| | about 100:1 | about 25:1 | 5:1 |
| [3-(2-methylaminoethyl)-1H-indol- | About 1:100 to | About 1:25 to | About 1:5 to about |
| 4-yl] dihydrogen phosphate | about 100:1 | about 25:1 | 5:1 |
| 4-hydroxy-N-methyltryptamine | About 1:100 to | About 1:25 to | About 1:5 to about |
| | about 100:1 | about 25:1 | 5:1 |
| [3-(aminoethyl)-1H-indol-4-yl] | About 1:100 to | About 1:25 to | About 1:5 to about |
| dihydrogen phosphate | about 100:1 | about 25:1 | 5:1 |
| [3-(2-trimethylaminoethyl)-1H- | About 1:100 to | About 1:25 to | About 1:5 to about |
| indol-4-yl] dihydrogen phosphate | about 100:1 | about 25:1 | 5:1 |
| 4-hydroxy-N,N,N- | About 1:100 to | About 1:25 to | About 1:5 to about |
| trimethyltryptamine | about 100:1 | about 25:1 | 5:1 |
| THC | About 1:100 to | About 1:25 to | About 1:5 to about |
| | about 100:1 | about 25:1 | 5:1 |
| CBC | About 1:100 to | About 1:25 to | About 1:5 to about |
| | about 100:1 | about 25:1 | 5:1 |
| CBD | About 1:100 to | About 1:25 to | About 1:5 to about |
| | about 100:1 | about 25:1 | 5:1 |
| CBG | About 1:100 to | About 1:25 to | About 1:5 to about |
| | about 100:1 | about 25:1 | 5:1 |
| Myrcene | About 1:100 to | About 1:25 to | About 1:5 to about |
| | about 100:1 | about 25:1 | 5:1 |
| Pinene | About 1:100 to | About 1:25 to | About 1:5 to about |
| | about 100:1 | about 25:1 | 5:1 |
| Caryophyllene | About 1:100 to | About 1:25 to | About 1:5 to about |
| | about 100:1 | about 25:1 | 5:1 |
| Limonene | About 1:100 to | About 1:25 to | About 1:5 to about |
| | about 100:1 | about 25:1 | 5:1 |
| Humulene | About 1:100 to | About 1:25 to | About 1:5 to about |
| | about 100:1 | about 25:1 | 5:1 |
| Linalool | About 1:100 to | About 1:25 to | About 1:5 to about |
| | about 100:1 | about 25:1 | 5:1 |
| Adrenaline | About 1:100 to | About 1:25 to | About 1:5 to about |
| | about 100:1 | about 25:1 | 5:1 |
| Amineptine | About 1:100 to | About 1:25 to | About 1:5 to about |
| | about 100:1 | about 25:1 | 5:1 |
| Erinacine A | About 1:100 to | About 1:25 to | About 1:5 to about |
| | about 100:1 | about 25:1 | 5:1 |
| Hericenone A | About 1:100 to | About 1:25 to | About 1:5 to about |
| | about 100:1 | about 25:1 | 5:1 |
| Phenelzine | About 1:100 to | About 1:25 to | About 1:5 to about |
| | about 100:1 | about 25:1 | 5:1 |

An “effective amount” or a “therapeutically effective amount” of a tryptammonium compound of the disclosure, a crystalline tryptammonium compound of the disclosure, or a specific crystalline form thereof, such as crystalline form 1 of a tryptammonium compound of the disclosure, is generally in the range of about 0.1 to about 100 mg daily (oral dose), of about 0.1 to about 50 mg daily (oral dose) of about 0.25 to about 25 mg daily (oral dose), of about 0.1 to about 5 mg daily (oral dose) or of about 0.5 to about 2.5 mg daily (oral dose). The actual amount required for treatment of any particular patient may depend upon a variety of factors including, for example, the disease being treated and its severity; the specific pharmaceutical composition employed; the age, body weight, general health, sex, and diet of the patient; the mode of administration; the time of administration; the route of administration; and the rate of excretion; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173 (2001), which is incorporated herein by reference. A tryptammonium compound of the disclosure, a crystalline tryptammonium compound of the disclosure, or a specific crystalline form thereof, such as crystalline form 1 of a tryptammonium compound of the disclosure, and pharmaceutical compositions containing it may be used in combination with other agents that are generally administered to a patient being treated for psychological and other disorders discussed above. They may also be co-formulated with one or more of such agents in a single pharmaceutical composition.

Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. Exemplary carriers include those that do not substantially alter the structure or activity of the tryptammonium compounds of the disclosure, crystalline tryptammonium compounds of the disclosure, or specific crystalline forms thereof, such as crystalline form 1 of a tryptammonium compound of the disclosure, nor produce undesirable biological effects or otherwise interact in a deleterious manner with any other component(s) of the pharmaceutical composition.

The pharmaceutical compositions of the disclosure may be prepared by methods know in the pharmaceutical formulation art, for example, see Remington's Pharmaceutical Sciences, 18th Ed., (Mack Publishing Company, Easton, Pa., 1990), which is incorporated herein by reference. In a solid dosage form, a 4-HO-DPT compound of the disclosure may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alignates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like, (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents. In some embodiments, the excipient is not water. In some embodiments, the excipient is not a solvent (e.g., EtOH, diethyl ether, ethyl acetate, or hydrocarbon-based solvents (e.g., hexanes). In some embodiments, the dosage form is substantially free of water and/or solvents, for example less than about 5% water by mass, less than 2% water by mass, less than 1% water by mass, less than 0.5% water by mass, or less than 0.1% water by mass.

Excipients or pharmaceutically acceptable adjuvants known in the pharmaceutical formulation art may also be used in the pharmaceutical compositions of the disclosure. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a pharmaceutical composition of the disclosure may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others well known in the art. They may contain pacifying agents and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Solid dosage forms for oral administration, which includes capsules, tablets, pills, powders, and granules, may be used. In such solid dosage forms, the active compound may be mixed with at least one inert, pharmaceutically acceptable excipient (also known as a pharmaceutically acceptable carrier).

Administration of tryptammonium compounds of the disclosure, crystalline tryptammonium compounds of the disclosure, or specific crystalline forms thereof, such as crystalline form 1 of a tryptammonium compound of the disclosure, in pure form or in an appropriate pharmaceutical composition may be carried out via any of the accepted modes of administration or agents for serving similar utilities. Thus, administration may be, for example, orally, buccally, nasally, parenterally (intravenous, intramuscular, or subcutaneous), topically, transdermally, intravaginally, intravesically, or intrasystemically, in the form of solid, semi-solid, lyophilized powder, or liquid dosage forms, such as, for example, tablets, suppositories, pills, soft elastic and hard gelatin capsules, powders, solutions, suspensions, or aerosols, or the like, such as, for example, in unit dosage forms suitable for simple administration of precise dosages. One route of administration may be oral administration, using a convenient daily dosage regimen that can be adjusted according to the degree of severity of the disease-state to be treated.

### Examples

### Synthesis and Crystallization

Compounds 1-methyltryptammonium chloride (1-Me-T·HCl), 1-phenyl-2-methyltryptammonium chloride (1-Ph-2-Me-T·HCl), and 5-methyltryptammonium chloride (5-Me-T·HCl) were purchased from Combi-Blocks, 5-chlorotryptammonium chloride (5-Cl-T·HCl) from Acela, 6-fluorotryptammonium chloride (6-F-T·HCl) from Chem Bridge, and 7-methyltryptammonium chloride (7-Me-T·HCl) from Frontier Scientific. The freebase of 5-methoxytryptammonium chloride (5-MeO-T·HCl) was purchased from TCI, of 5-bromotryptammonium chloride (5-Br-T·HCl) from Alfa Aesar, and 5-fluorotryptammonium chloride (5-F-T·HCl) from Combi-Blocks. The freebase compounds were dissolved in methylene chloride and converted to the hydrochloride salt by adding a three-fold excess of HCl via a 2M solution in diethyl ether. The resulting precipitates were filtered and washed with hexanes to produce their HCl salts. Crystals suitable for X-ray diffraction studies were grown from the slow evaporation of solutions of each compound. The solvents used in each case were: a ethyl acetate/tetrahydrofuran/methanol mixture for 1-Me-T·HCl, an acetone/isopropanol/toluene mixture for 1-Ph-2-Me-T·HCl, acetone for 5-MeO-T·HCl, an acetone/water mixture for 5-Br-T·HCl, an acetone/isopropanol mixture for 5-Cl-T·HCl, water for 5-F-T·HCl, isopropanol for 5-Me-T·HCl, methanol for 6-F-T·HCl, and an acetonitrile/methanol mixture for 7-Me-T·HCl.

### Refinement

Crystal data, data collection, and structure refinement details for 1-Me-T·HCl, 1-Ph-2-Me-T·HCl, 5-MeO-T·HCl, and 5-Br-T·HCl are summarized in Table 3. Crystal data, data collection, and structure refinement details for 5-Cl-T·HCl, 5-F-T·HCl, 5-Me-T·HCl, and 6-F-T·HCl are summarized in Table 4. Crystal data, data collection, and structure refinement details for 7-Me-T·HCl are summarized in Table 5. Hydrogen atoms attached to ammonium nitrogen atoms were found from a difference-Fourier map and were refined isotropically, using DFIX restraints with N-H distances of 0.99 (1) A. Hydrogen atoms attached to indole nitrogen atoms were similarly found from a difference—Fourier map and were refined isotropically, using DFIX restraints with N-H distances of 0.87 (1) A. Isotropic displacement parameters were set to 1.2U_{eq} of the parent indole nitrogen and 1.5U_{eq} of the parent ammonium nitrogen atom. All other hydrogen atoms were placed in calculated positions (C-H=0.93-0.97 A). Isotropic displacement parameters were set to 1.2U_{eq}(C) or 1.5U_{eq}(C-methyl).

TABLE 3

| | (1-Me-T · HCl) | (1-Ph-2-Me-T · HCl) | (5-MeO-T · HCl) | (5-Br-T · HCl) |
|---|---|---|---|---|
| Crystal data | | | | |
| Chemical formula | C₁₁H₁₅N₂ · Cl | C₁₇H₁₉N₂ · Cl | C₁₁H₁₅N₂O · Cl | C₁₀H₁₂BrN₂ · Cl |
| Mᵣ | 210.70 | 286.79 | 226.70 | 275.58 |
| Crystal system, space | Trigonal, R3c | Monoclinic, P2₁/n | Monoclinic, P2₁/c | Orthorhombic, |
| group | | | | Pbca |
| Temperature (K) | 297 | 297 | 297 | 297 |
| a, b, c (Å) | 29.3337 (13), 29.3337 | 10.3990 (6), 16.3016 | 14.6858 (8), 8.3613 | 8.6153 (6), 9.3766 |
| | (13), 7.3922 (6) | (10), 37.091 (2) | (4), 9.7878 (5) | (5), |
| | | | | 29.173 (2) |
| α, β, γ (°) | 90, 90, 120 | 90, 97.963 (2), 90 | 90, 102.742 (2), 90 | 90, 90, 90 |
| V (Å³) | 5508.6 (7) | 6227.1 (7) | 1172.27 (10) | 2356.7 (3) |
| Z | 18 | 16 | 4 | 8 |
| Radiation type | Mo Kα | Mo Kα | Mo Kα | Mo Kα |
| μ (mm⁻¹) | 0.28 | 0.24 | 0.30 | 3.68 |
| Crystal size (mm) | 0.31 × 0.08 × 0.07 | 0.24 × 0.20 × 0.10 | 0.24 × 0.21 × 0.04 | 0.28 × 0.21 × 0.12 |
| Data collection | | | | |
| Diffractometer | Bruker D8 Venture | Bruker D8 Venture | Bruker D8 Venture | Bruker D8 Venture |
| | CMOS | CMOS | CMOS | CMOS |
| Absorption correction | Multi-scan | Multi-scan | Multi-scan | Multi-scan |
| | SADABS2016/2 | SADABS2016/2 | SADABS2016/2 | SADABS2016/2 |
| | (Bruker, 2016/2) was | (Bruker, 2016/2) was | (Bruker, 2016/2) was | (Bruker, 2016/2) |
| | used forabsorption | used forabsorption | used forabsorption | was used for |
| | correction. wR2(int) | correction. wR2(int) | correction. wR2(int) | absorption |
| | was 0.0574 beforeand | was 0.0531 before and | was 0.0586 before | correction. |
| | 0.0514 after | 0.0493 after correction. | and 0.0527 after | wR2(int) was |
| | correction. The Ratio of | The Ratio of minimum | correction. The Ratio | 0.0850 before and |
| | minimum to maximum | to maximum | of minimum to | 0.0484 after |
| | transmission is 0.9466. | transmission is 0.9569. | maximum | correction. The |
| | The λ/2 correction | The λ/2 correction | transmission is | Ratio of minimum |
| | factor is Not present. | factoris Not present. | 0.9341. The λ/2 | to maximum |
| | | | correction factoris | transmission is |
| | | | Not present. | 0.7474. The λ/2 |
| | | | | correction factoris |
| | | | | Not present. |
| Tₘᵢₙ, Tₘₐₓ | 0.706, 0.745 | 0.713, 0.745 | 0.696, 0.745 | 0.483, 0.647 |
| No. of measured, | 32453, 2315, 2091 | 111482, 11846, 8101 | 21284, 2213, 1842 | 55777, 2377, 1900 |
| independent and | | | | |
| observed [I > 2σ(I)] | | | | |
| reflections | | | | |
| Rᵢₙₜ | 0.041 | 0.057 | 0.040 | 0.047 |
| (sin θ/λ)ₘₐₓ (Å⁻¹) | 0.610 | 0.612 | 0.611 | 0.624 |
| Refinement | | | | |
| R[F² > 2σ(F²)], wR(F²), S | 0.038, 0.103, 1.03 | 0.054, 0.130, 1.08 | 0.035, 0.098, 1.03 | 0.047, 0.096, 1.08 |
| No. of reflections | 2315 | 11846 | 2213 | 2377 |
| No. of parameters | 140 | 773 | 149 | 143 |
| No. of restraints | 4 | 12 | 4 | 4 |
| H-atom treatment | H atoms treated by a | H atoms treated by a | H atoms treated by a | H atoms treated by a |
| | mixtureof | mixture of | mixture of | mixture of |
| | independent and | independentand | independentand | independentand |
| | constrained | constrained | constrained | constrained |
| | refinement | refinement | refinement | refinement |
| Δρₘₐₓ, Δρₘᵢₙ (e Å⁻³) | 0.60, −0.15 | 0.19, −0.23 | 0.18, −0.24 | 0.55, −0.72 |
| Absolute structure | Flack × determined | — | — | — |
| | using 922 quotients | | | |
| | [(I+) − (I−)]/[(I+) + (I−)] | | | |
| | (Parsons, Flack and | | | |
| | Wagner, Acta Cryst. | | | |
| | B69 (2013) 249-259). | | | |
| Absolute structure | 0.009 (18) | — | — | — |
| parameter | | | | |

TABLE 4

| | (5-Cl-T · HCl) | (5-F-T · HCl) | (5-Me-T · HCl) | (6-F-T · HCl) |
|---|---|---|---|---|
| Crystal data | | | | |
| Chemical formula | C₁₀H₁₂ClN₂ · Cl | C₁₀H₁₂FN₂ · Cl | C₁₁H₁₅N₂ · Cl | C₁₀H₁₂FN₂ · Cl |
| Mᵣ | 231.12 | 214.67 | 210.70 | 214.67 |
| Crystal system, space | Monoclinic, P2₁/c | Orthorhombic, Pbca | Monoclinic, P2₁/c | Orthorhombic, Pbca |
| group | | | | |
| Temperature (K) | 297 | 297 | 297 | 297 |
| a, b, c (Å) | 14.7030 (9), 8.6058 | 8.6708 (4), 9.6684 | 14.9939 (10), 8.4270 | 8.3572 (4), 10.3493 |
| | (5), | (5), | (5), 9.5388 (6) | (5), |
| | 9.4141 (5) | 25.6854 (12) | | 24.3824 (13) |
| α, β, γ (°) | 90, 106.450 (2), 90 | 90, 90, 90 | 90, 107.774 (2), 90 | 90,90,90 |
| V (Å³) | 1142.42 (11) | 2153.28 (18) | 1147.73 (13) | 2108.86 (18) |
| Z | 4 | 8 | 4 | 8 |
| Radiation type | Mo Kα | Mo Kα | Mo Kα | Mo Kα |
| μ (mm⁻¹) | 0.53 | 0.33 | 0.30 | 0.34 |
| Crystal size (mm) | 0.24 × 0.20 × 0.06 | 0.29 × 0.20 × 0.03 | 0.22 × 0.20 × 0.08 | 0.35 × 0.24 × 0.06 |
| Data collection | | | | |
| Diffractometer | Bruker D8 Venture | Bruker D8 Venture | Bruker D8 Venture | Bruker D8 Venture |
| | CMOS | CMOS | CMOS | CMOS |
| Absorption | Multi-scan | Multi-scan | Multi-scan | Multi-scan |
| correction | SADABS2016/2 | SADABS2016/2 | SADABS2016/2 | SADABS2016/2 |
| | (Bruker, 2016/2) | (Bruker, 2016/2) | (Bruker, 2016/2) | (Bruker, 2016/2) |
| | was used for | was used for | was used for | was usedfor |
| | absorption | absorption | absorption | absorption |
| | correction. | correction. | correction. | correction. |
| | wR2(int) was | wR2(int) was | wR2(int) was | wR2(int) was |
| | 0.0550 before and | 0.0588 before and | 0.0612 before and | 0.0561 before |
| | 0.0487 after | 0.0528 after | 0.0539 after | and 0.0495 after |
| | correction. The | correction. The | correction. The | correction. The |
| | Ratio of minimum | Ratio of minimum | Ratio of minimum | Ratio of minimum |
| | to maximum | to maximum | to maximum | to maximum |
| | transmission is | transmission is | transmission is | transmission is |
| | 0.9462. | 0.9310. | 0.9101. | 0.9223. |
| | The λ/2 correction | The λ/2 correction | The λ/2 correction | The λ/2 |
| | factor is Not | factor is Not | factor is Not | correction factor |
| | present. | present. | present. | is Not present. |
| Tₘᵢₙ, Tₘₐₓ | 0.705, 0.745 | 0.694, 0.745 | 0.678, 0.745 | 0.688, 0.745 |
| No. of measured, | 27018, 2315, 1858 | 65854, 2190, 1904 | 25382, 2331, 1932 | 56982, 2154, 1922 |
| independent and | | | | |
| observed [I > 2σ(I)] | | | | |
| reflections | | | | |
| Rᵢₙₜ | 0.039 | 0.043 | 0.043 | 0.034 |
| (sin θ/λ)max (Å⁻¹) | 0.626 | 0.625 | 0.626 | 0.625 |
| Refinement | | | | |
| R[F² > 2σ(F²)], wR(F²), | 0.036, 0.087, 1.05 | 0.041, 0.099, 1.08 | 0.049, 0.148, 1.15 | 0.037, 0.093, 1.15 |
| S | | | | |
| No. of reflections | 2315 | 2190 | 2331 | 2154 |
| No. of parameters | 143 | 143 | 144 | 143 |
| No. of restraints | 4 | 4 | 4 | 4 |
| H-atom treatment | H atoms treated by a | H atoms treated by a | H atoms treated by a | H atoms treated by a |
| | mixture of | mixture of | mixture of | mixture of |
| | independent and | independent and | independent and | independent and |
| | constrained | constrained | constrained | constrained |
| | refinement | refinement | refinement | refinement |
| Δρₘₐₓ, Δρₘᵢₙ (e Å⁻³) | 0.24, −0.29 | 0.20, −0.19 | 0.34, −0.30 | 0.18, −0.20 |

TABLE 5

| | (7-Me-T · HCl) |
|---|---|
| Crystal data | |
| Chemical formula | C₁₁H₁₅N₂ · Cl |
| Mᵣ | 210.70 |
| Crystal system, space | Orthorhombic, Pbca |
| group | |
| Temperature (K) | 297 |
| a, b, c (Å) | 9.1893 (5), 9.3259 |
| | (4), 27.5149 (15) |
| α, β, γ (°) | 90, 90, 90 |
| V (Å³) | 2358.0 (2) |
| Z | 8 |
| Radiation type | Mo Kα |
| μ (mm⁻¹) | 0.29 |
| Crystal size (mm) | 0.24 × 0.16 × 0.02 |
| Data collection | |
| Diffractometer | Bruker D8 Venture |
| | CMOS |
| Absorption correction | Multi-scan |
| | SADABS2016/2 |
| | (Bruker, 2016/2) |
| | was used for |
| | absorption |
| | correction. |
| | wR2(int) was |
| | 0.0575 before and |
| | 0.0531 after |
| | correction. The |
| | Ratio of minimum |
| | to maximum |
| | transmission is |
| | 0.9434. The λ/2 |
| | correction factor is |
| | Not present. |
| Tₘᵢₙ, Tₘₐₓ | 0.703, 0.745 |
| No. of measured, | 43558, 2237, 1791 |
| independent and | |
| observed [I > 2σ(I)] | |
| reflections | |
| Rᵢₙₜ | 0.054 |
| (sin θ/λ)ₘₐₓ (Å⁻¹) | 0.610 |
| Refinement | |
| R[F² > 2σ(F²)], wR(F²), S | 0.039, 0.091, 1.06 |
| No. of reflections | 2237 |
| No. of parameters | 144 |
| No. of restraints | 4 |
| H-atom treatment | H atoms treated by |
| | a mixture of |
| | independent and |
| | constrained |
| Δρₘₐₓ, Δρₘᵢₙ (e Å⁻³) | 0.15, −0.13 |

Computer programs: APEX3 (Bruker, 2018), SAINT (Bruker, 2018), SHELXT2014 (Sheldrick, 2015a), SHELXL2018 (Sheldrick, 2015b), OLEX2 (Dolomanov et al., 2009), pub/CIF (Westrip, 2010).

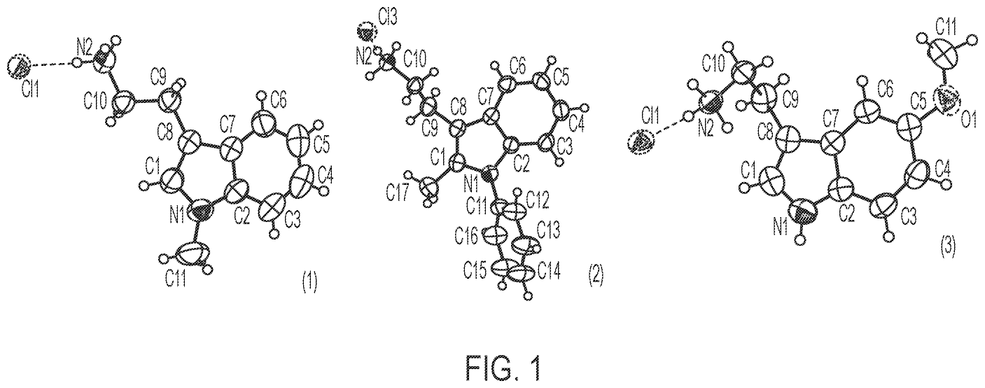

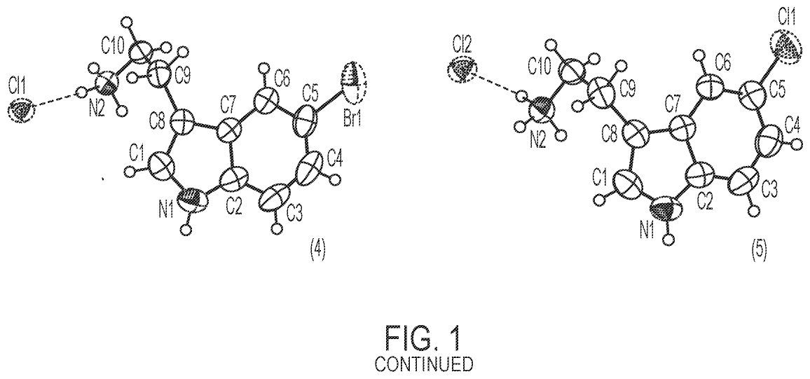

The molecular structure, showing the atomic labeling, of the following compounds: (1) 1-methyltryptammonium chloride (1-Me-T·HCl), (2) 1-phenyl-2-methyltryptammonium chloride (1-Ph-2-Me-T·HCl), (3) 5-methoxytryptammonium chloride (5-MeO-T·HCl), (4) 5-bromotryptammonium chloride (5-Br-T·HCl), (5) 5-chlorotryptammonium chloride (5-Cl-T·HCl), (6) 5-fluorotryptammonium chloride (5-F-T·HCl), (7) 5-methyltryptammonium chloride (5-Me-T·HCl), (8) 6-fluorotryptammonium chloride (6-F-T·HCl), and (9) 7-methyltryptammonium chloride (7-Me-T·HCl), is shown in FIG. 1. Displacement ellipsoids are drawn at the 50% probability level. Hydrogen bonds are shown as dashed lines.

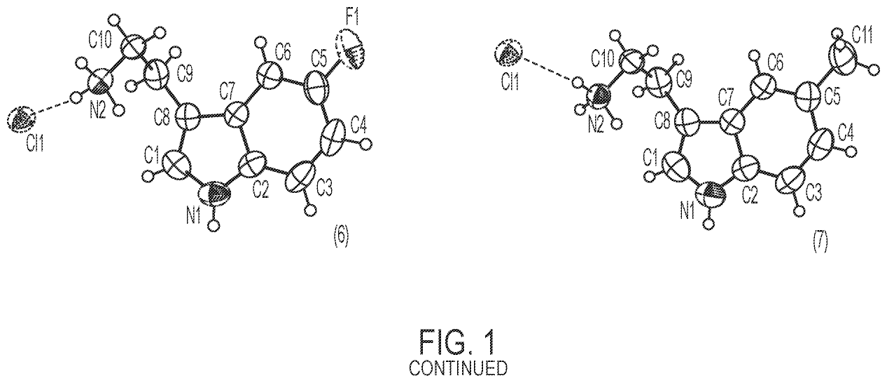

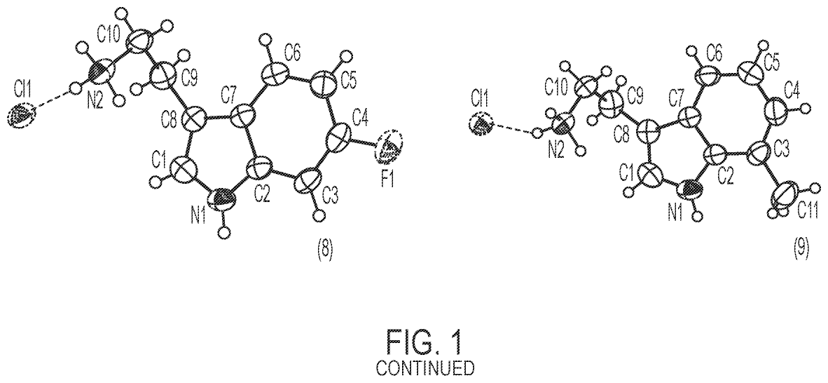

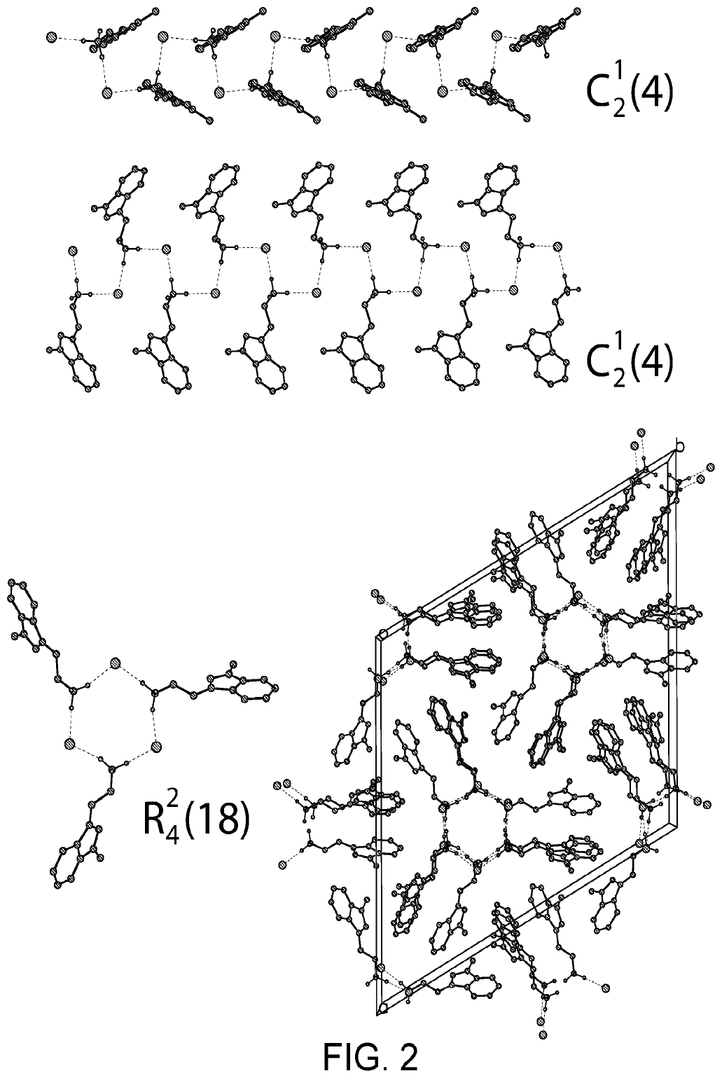

FIG. 2 depicts the crystal packing of crystalline form 1 of 1-methyltryptammonium chloride along the c-axis (bottom right), which shows the one-dimensional hydrogen bonding network along [001]. This network consists of R²₄ (18) rings and C¹₂ (4) chains, shown in the figure. Hydrogens not involved in hydrogen bonding have been removed for clarity.

FIG. 3 depicts the crystal packing of crystalline form 1 of 1-phenyl-2-methyltryptammonium chloride along the a-axis (bottom right), which shows the two-dimensional hydrogen bonding network along [100]. This network consists of R⁴₆(12) and R⁴₈(16) rings and C²⁴(8) chains, shown in the figure.

Hydrogens not involved in hydrogen bonding have been removed for clarity.

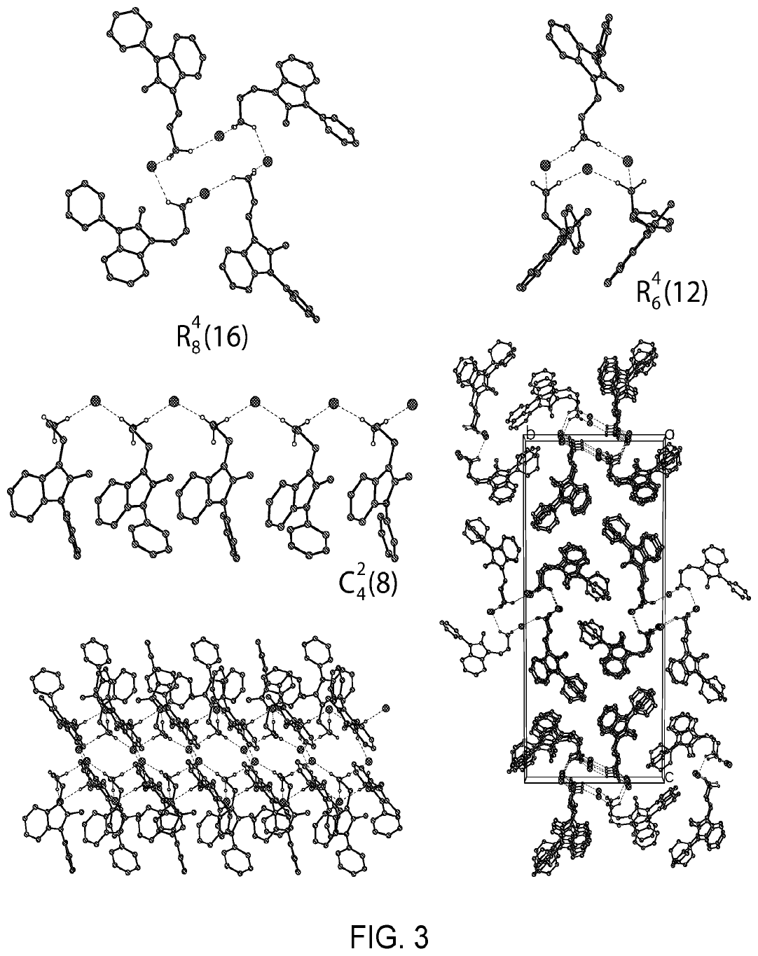

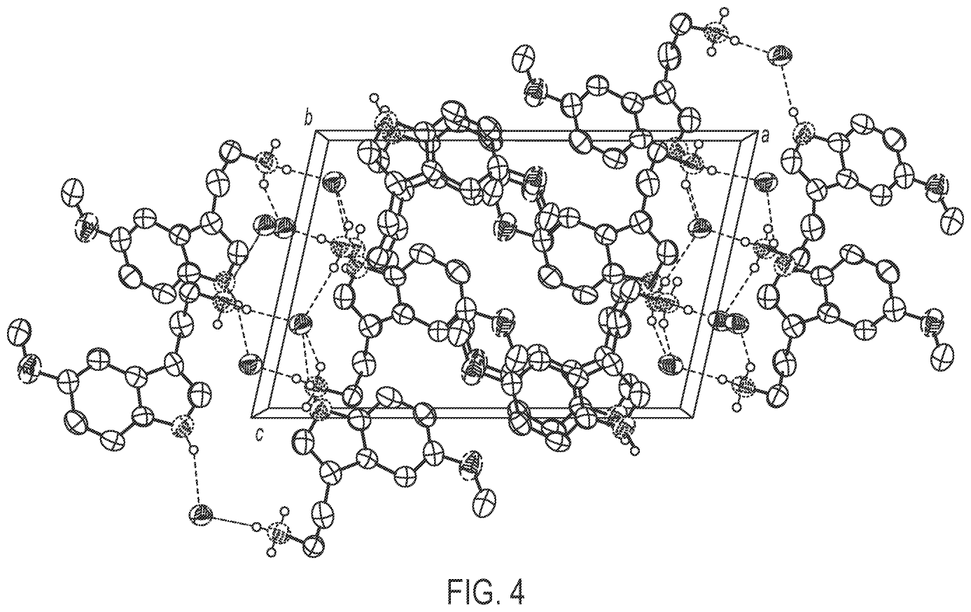

FIG. 4 depicts the crystal packing of crystalline form 1 of 5-methoxytryptammonium chloride along the b-axis.

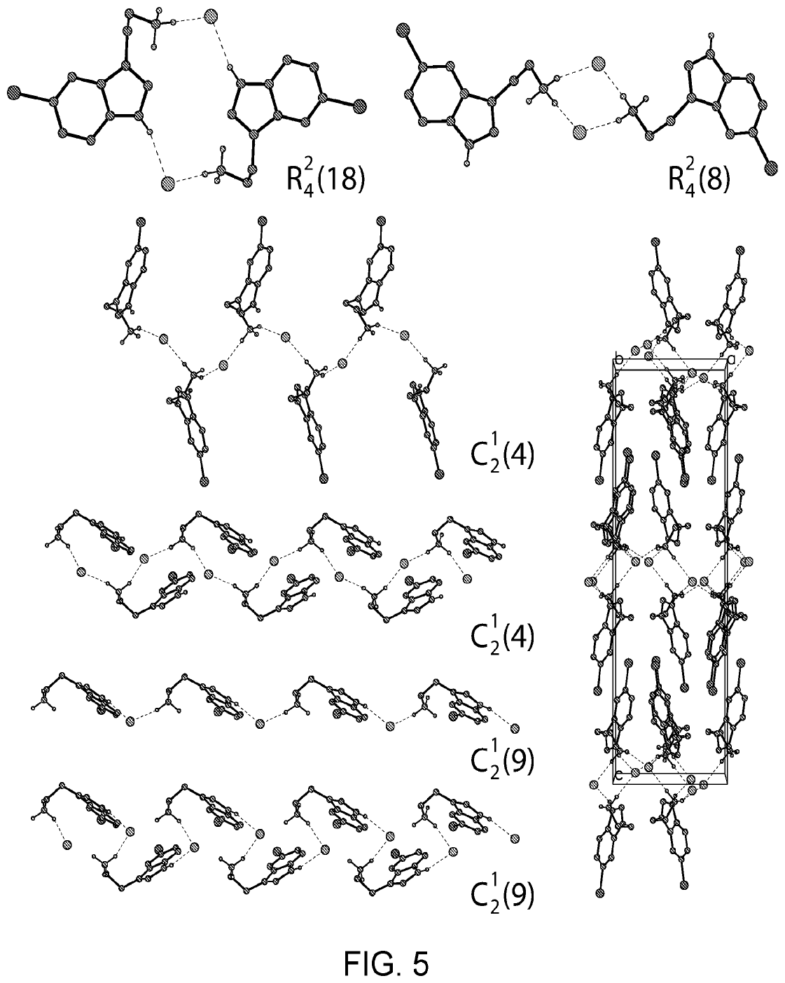

FIG. 5 depicts the crystal packing of crystalline form 1 of 5-bromotryptammonium chloride along the b-axis (bottom right), which shows the two-dimensional hydrogen bonding network along (001). This network consists of R²₄ (8) and R²₄ (18) rings and C¹₂ (4) and C¹₂ (9) chains, shown in the figure. Hydrogens not involved in hydrogen bonding have been removed for clarity.

FIG. 6 depicts the crystal packing of crystalline form 1 of 5-chlorotryptammonium chloride along the c-axis.

FIG. 7 depicts the crystal packing of crystalline form 1 of 5-fluorotryptammonium chloride along the b-axis.

FIG. 8 depicts the crystal packing of crystalline form 1 of 5-methyltryptammonium chloride along the c-axis (bottom right), which shows the two-dimensional hydrogen bonding network along (100). This network consists of R²₄ (8) and R²₄ (18) rings and C¹₂ (4) and C¹₂ (9) chains, shown in the figure. Hydrogens not involved in hydrogen bonding have been removed for clarity.

FIG. 9 depicts the crystal packing of crystalline form 1 of 6-fluorotryptammonium chloride along the b-axis.

FIG. 10 depicts the crystal packing of crystalline form 1 of 7-methyltryptammonium chloride along the b-axis.

FIG. 11 shows a simulated X-ray powder diffraction pattern (XRPD) for crystalline form 1 of 1-methyltryptammonium chloride generated from its single crystal data. Table 6 lists the angles (° 2θ±0.2 ° 2θ) and d-spacing of the peaks identified in the experimental XRPD pattern of FIG. 11. The entire list of peaks, or a subset thereof, may be sufficient to characterize the cocrystal. For example, the cocrystal may be characterized by at least two peaks selected from the peaks at 6.0, 10.4, 15.1, 16.0, and 19.4 ° 2θ±0.2 ° 2θ or their corresponding d-spacing as well as by an XRPD pattern substantially similar to FIG. 11.

FIG. 12 shows a simulated X-ray powder diffraction pattern (XRPD) for crystalline form 1 of 1-phenyl-2-methyltryptammonium chloride generated from its single crystal data. Table 7 lists the angles (^{°} 2θ±0.2 ° 2θ) and d-spacing of the peaks identified in the experimental XRPD pattern of FIG. 12. The entire list of peaks, or a subset thereof, may be sufficient to characterize the cocrystal. For example, the cocrystal may be characterized by at least two peaks selected from the peaks at 9.0, 15.4, and 17.2 ° 2θ±0.2 ° 2θ or their corresponding d-spacing as well as by an XRPD pattern substantially similar to FIG. 12.

FIG. 13 shows a simulated X-ray powder diffraction pattern (XRPD) for crystalline form 1 of 5-methoxytryptammonium chloride generated from its single crystal data. Table 8 lists the angles (° 2θ±0.2 ° 2θ) and d-spacing of the peaks identified in the experimental XRPD pattern of FIG. 13. The entire list of peaks, or a subset thereof, may be sufficient to characterize the cocrystal. For example, the cocrystal may be characterized by at least two peaks selected from the peaks at 14.1, 16.2, and 22.6 ° 2θ±0.2 ° 2θ or their corresponding d-spacing as well as by an XRPD pattern substantially similar to FIG. 13.

FIG. 14 shows a simulated X-ray powder diffraction pattern (XRPD) for crystalline form 1 of 5-bromotryptammonium chloride generated from its single crystal data. Table 9 lists the angles (° 2θ±0.2 ° 2θ) and d-spacing of the peaks identified in the experimental XRPD pattern of FIG. 14. The entire list of peaks, or a subset thereof, may be sufficient to characterize the cocrystal. For example, the cocrystal may be characterized by at least two peaks selected from the peaks at 6.1, 16.7, and 19.9 ° 2θ±0.2 ° 2θ or their corresponding d-spacing as well as by an XRPD pattern substantially similar to FIG. 14.

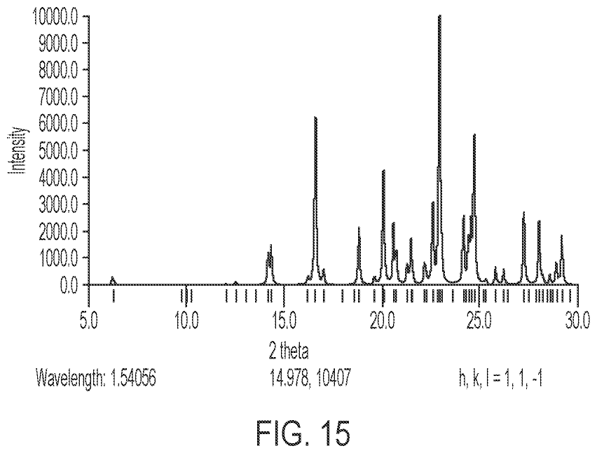

FIG. 15 shows a simulated X-ray powder diffraction pattern (XRPD) for crystalline form 1 of 5-chlorotryptammonium chloride generated from its single crystal data. Table 10 lists the angles (° 2θ±0.2 ° 2θ) and d-spacing of the peaks identified in the experimental XRPD pattern of FIG. 15. The entire list of peaks, or a subset thereof, may be sufficient to characterize the cocrystal. For example, the cocrystal may be characterized by at least two peaks selected from the peaks at 16.6, 20.1, and 23.0 ° 2θ±0.2 ° 2θ or their corresponding d-spacing as well as by an XRPD pattern substantially similar to FIG. 15.

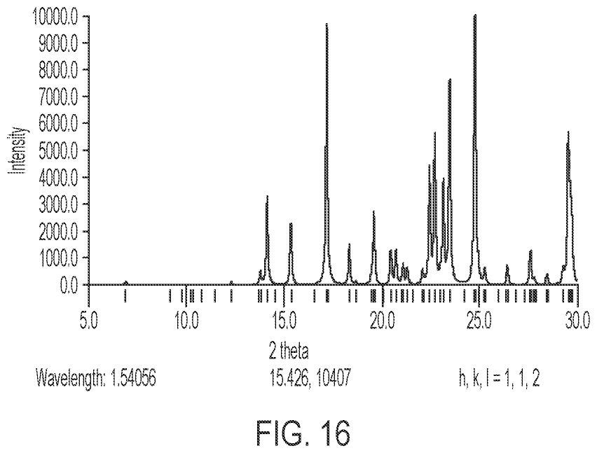

FIG. 16 shows a simulated X-ray powder diffraction pattern (XRPD) for crystalline form 1 of 5-fluorotryptammonium chloride generated from its single crystal data. Table 11 lists the angles (° 2θ±0.2 ° 2θ) and d-spacing of the peaks identified in the experimental XRPD pattern of FIG. 16. The entire list of peaks, or a subset thereof, may be sufficient to characterize the cocrystal. For example, the cocrystal may be characterized by at least two peaks selected from the peaks at 14.1, 15.3, and 17.2 ° 2θ±0.2 ° 2θ or their corresponding d-spacing as well as by an XRPD pattern substantially similar to FIG. 16.

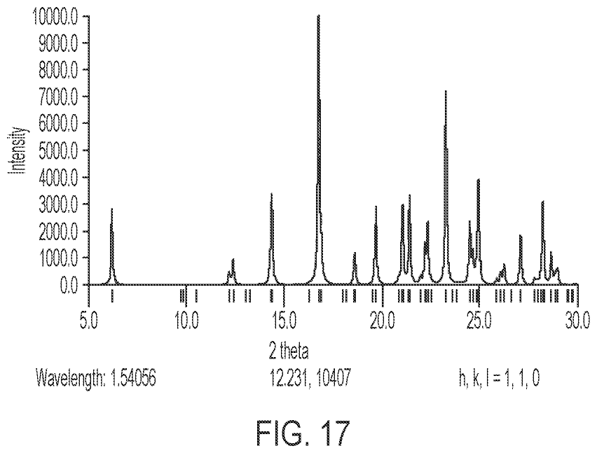

FIG. 17 shows a simulated X-ray powder diffraction pattern (XRPD) for crystalline form 1 of 5-methyltryptammonium chloride generated from its single crystal data. Table 12 lists the angles (° 2θ±0.2 ° 2θ) and d-spacing of the peaks identified in the experimental XRPD pattern of FIG. 17. The entire list of peaks, or a subset thereof, may be sufficient to characterize the cocrystal. For example, the cocrystal may be characterized by at least two peaks selected from the peaks at 6.2, 19.7, and 23.3 ° 2θ±0.2 ° 2θ or their corresponding d-spacing as well as by an XRPD pattern substantially similar to FIG. 17.

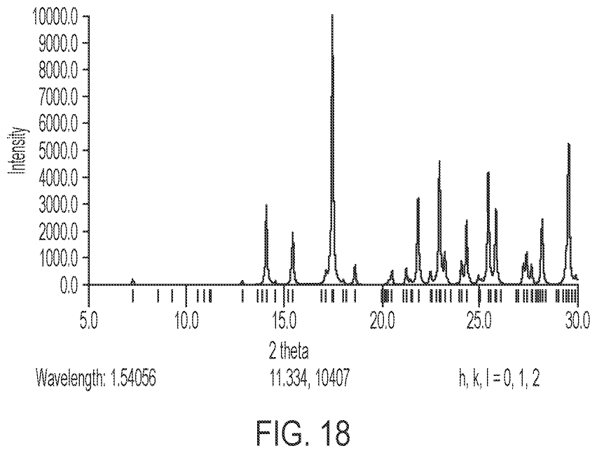

FIG. 18 shows a simulated X-ray powder diffraction pattern (XRPD) for crystalline form 1 of 6-fluorotryptammonium chloride generated from its single crystal data. Table 13 lists the angles (° 2θ±0.2 ° 2θ) and d-spacing of the peaks identified in the experimental XRPD pattern of FIG. 18. The entire list of peaks, or a subset thereof, may be sufficient to characterize the cocrystal. For example, the cocrystal may be characterized by at least two peaks selected from the peaks at 14.1, 15.4, and 21.9 ° 2θ±0.2 ° 2θ or their corresponding d-spacing as well as by an XRPD pattern substantially similar to FIG. 18.

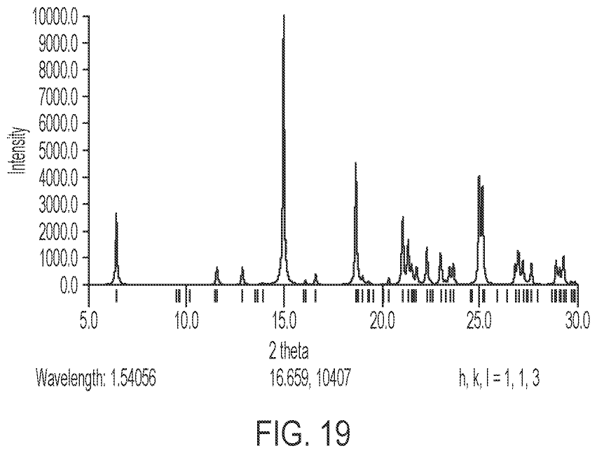

FIG. 19 shows a simulated X-ray powder diffraction pattern (XRPD) for crystalline form 1 of 7-methyltryptammonium chloride generated from its single crystal data. Table 14 lists the angles (° 2θ±0.2 ° 2θ) and d-spacing of the peaks identified in the experimental XRPD pattern of FIG. 19. The entire list of peaks, or a subset thereof, may be sufficient to characterize the cocrystal. For example, the cocrystal may be characterized by at least two peaks selected from the peaks at 6.4, 15.0, and 18.7 ° 2θ±0.2 ° 2θ or their corresponding d-spacing as well as by an XRPD pattern substantially similar to FIG. 19.

TABLE 6
1-Me-T•HCl

| d-spacing (Å) | °2θ ± 0.2°2θ | Intensity |
|---|---|---|
| 14.67 | 6.02 | 11395 |
| 8.47 | 10.44 | 58339 |
| 7.33 | 12.06 | 1325 |
| 5.86 | 15.11 | 331444 |
| 5.54 | 15.97 | 182275 |
| 5.10 | 17.37 | 713034 |
| 4.89 | 18.13 | 826590 |
| 4.58 | 19.38 | 861948 |
| 4.23 | 20.96 | 69212 |
| 4.07 | 21.83 | 181553 |
| 4.03 | 22.06 | 413036 |
| 3.88 | 22.89 | 86396 |
| 3.67 | 24.25 | 571350 |
| 3.66 | 24.31 | 774336 |
| 3.64 | 24.46 | 1031400 |
| 3.55 | 25.07 | 85259 |
| 3.45 | 25.81 | 47444 |
| 3.43 | 25.94 | 361904 |
| 3.36 | 26.47 | 256156 |
| 3.27 | 27.22 | 182291 |
| 3.26 | 27.35 | 153373 |
| 3.20 | 27.85 | 33725 |
| 3.19 | 27.91 | 231485 |
| 3.18 | 28.03 | 12330 |
| 3.12 | 28.57 | 62403 |
| 2.99 | 29.87 | 63199 |
| 2.98 | 29.99 | 214489 |

TABLE 7
1-Ph-2-Me-T•HCl

| d-spacing (Å) | °2θ ± 0.2°2θ | Intensity |
|---|---|---|
| 14.90 | 5.93 | 1045 |
| 12.19 | 7.24 | 496 |
| 10.29 | 8.58 | 248 |
| 9.79 | 9.03 | 2175 |
| 9.58 | 9.23 | 68 |
| 9.18 | 9.62 | 65427 |
| 8.71 | 10.15 | 668 |
| 8.70 | 10.15 | 734 |
| 8.48 | 10.42 | 535 |
| 8.26 | 10.70 | 1071 |
| 8.25 | 10.71 | 783 |
| 8.15 | 10.85 | 63723 |
| 8.00 | 11.05 | 110203 |
| 7.96 | 11.11 | 85750 |
| 7.53 | 11.74 | 751 |
| 7.52 | 11.75 | 10 |
| 7.45 | 11.87 | 44614 |
| 7.39 | 11.96 | 806 |
| 6.78 | 13.04 | 79332 |
| 6.73 | 13.14 | 2936 |
| 6.72 | 13.16 | 549 |
| 6.70 | 13.21 | 17716 |
| 6.42 | 13.79 | 173 |
| 6.39 | 13.84 | 100 |
| 6.39 | 13.85 | 2310 |
| 6.21 | 14.26 | 16 |
| 6.20 | 14.26 | 103 |
| 6.12 | 14.46 | 13 |
| 6.10 | 14.52 | 8361 |
| 5.98 | 14.81 | 147 |
| 5.97 | 14.83 | 2047 |
| 5.88 | 15.05 | 4214 |
| 5.88 | 15.06 | 480 |
| 5.73 | 15.45 | 204656 |
| 5.62 | 15.74 | 186 |
| 5.48 | 16.17 | 3447 |
| 5.47 | 16.19 | 4225 |
| 5.46 | 16.23 | 434404 |
| 5.38 | 16.48 | 68660 |
| 5.32 | 16.66 | 9 |
| 5.31 | 16.68 | 53 |
| 5.21 | 17.00 | 12311 |
| 5.15 | 17.21 | 35831 |
| 5.15 | 17.21 | 244134 |
| 5.05 | 17.56 | 4 |
| 5.04 | 17.58 | 27 |
| 5.00 | 17.74 | 4459 |
| 4.97 | 17.84 | 36751 |
| 4.96 | 17.86 | 5755 |
| 4.95 | 17.89 | 114352 |
| 4.91 | 18.05 | 16426 |
| 4.91 | 18.06 | 27887 |
| 4.90 | 18.11 | 222660 |
| 4.81 | 18.45 | 794 |
| 4.81 | 18.45 | 535 |
| 4.79 | 18.51 | 70053 |
| 4.78 | 18.53 | 645 |
| 4.78 | 18.54 | 219608 |
| 4.78 | 18.54 | 99394 |
| 4.75 | 18.65 | 11159 |
| 4.75 | 18.68 | 1021 |
| 4.73 | 18.76 | 4596 |
| 4.72 | 18.77 | 1533 |
| 4.68 | 18.96 | 2726 |
| 4.63 | 19.16 | 11570 |
| 4.62 | 19.18 | 7962 |
| 4.59 | 19.30 | 10493 |
| 4.59 | 19.31 | 3149 |
| 4.59 | 19.32 | 56118 |
| 4.58 | 19.38 | 1942 |
| 4.58 | 19.38 | 5480 |
| 4.43 | 20.01 | 4629 |
| 4.42 | 20.07 | 260 |
| 4.41 | 20.11 | 79208 |
| 4.38 | 20.24 | 67210 |
| 4.38 | 20.27 | 3422 |
| 4.38 | 20.28 | 2166 |
| 4.37 | 20.31 | 25996 |
| 4.36 | 20.34 | 29245 |
| 4.36 | 20.37 | 10377 |
| 4.35 | 20.38 | 427952 |
| 4.35 | 20.39 | 5279 |
| 4.28 | 20.74 | 527 |
| 4.27 | 20.77 | 2751 |
| 4.27 | 20.81 | 326057 |
| 4.26 | 20.82 | 71186 |
| 4.25 | 20.90 | 21858 |
| 4.24 | 20.92 | 807 |
| 4.24 | 20.93 | 1513 |
| 4.24 | 20.94 | 676 |
| 4.15 | 21.40 | 1898 |
| 4.15 | 21.41 | 1552 |
| 4.13 | 21.50 | 35384 |
| 4.13 | 21.52 | 3871 |
| 4.11 | 21.60 | 46108 |
| 4.10 | 21.64 | 134293 |
| 4.08 | 21.79 | 4026 |
| 4.06 | 21.85 | 42110 |
| 4.05 | 21.93 | 14559 |
| 4.00 | 22.20 | 199440 |
| 3.99 | 22.27 | 611 |
| 3.98 | 22.33 | 93556 |
| 3.96 | 22.44 | 7175 |
| 3.96 | 22.44 | 48755 |
| 3.95 | 22.47 | 121673 |
| 3.91 | 22.74 | 6334 |
| 3.90 | 22.75 | 104 |
| 3.89 | 22.81 | 7497 |
| 3.89 | 22.84 | 8196 |
| 3.88 | 22.91 | 9003 |
| 3.87 | 22.94 | 5946 |
| 3.87 | 22.98 | 130003 |
| 3.85 | 23.06 | 359746 |
| 3.85 | 23.09 | 1 |
| 3.79 | 23.46 | 45 |
| 3.79 | 23.46 | 83 |
| 3.77 | 23.55 | 4737 |
| 3.77 | 23.60 | 11602 |
| 3.76 | 23.63 | 163046 |
| 3.76 | 23.66 | 55873 |
| 3.75 | 23.71 | 159 |
| 3.75 | 23.71 | 590 |
| 3.74 | 23.79 | 76748 |
| 3.74 | 23.79 | 36535 |
| 3.73 | 23.87 | 17470 |
| 3.70 | 24.05 | 354 |
| 3.69 | 24.10 | 624440 |
| 3.68 | 24.16 | 543488 |
| 3.68 | 24.17 | 30644 |
| 3.67 | 24.20 | 361 |
| 3.67 | 24.21 | 309 |
| 3.67 | 24.21 | 12826 |
| 3.67 | 24.25 | 3098 |
| 3.66 | 24.27 | 21951 |
| 3.65 | 24.37 | 55369 |
| 3.63 | 24.53 | 4752 |
| 3.61 | 24.67 | 26066 |
| 3.60 | 24.71 | 446184 |
| 3.59 | 24.76 | 405608 |
| 3.59 | 24.78 | 12084 |
| 3.59 | 24.81 | 8281 |
| 3.58 | 24.83 | 22255 |
| 3.58 | 24.83 | 131 |
| 3.57 | 24.93 | 74 |
| 3.57 | 24.94 | 1411 |
| 3.57 | 24.95 | 1097 |
| 3.56 | 24.96 | 259698 |
| 3.56 | 24.98 | 26536 |
| 3.54 | 25.14 | 2593 |
| 3.54 | 25.16 | 5274 |
| 3.51 | 25.37 | 1738 |
| 3.48 | 25.59 | 100486 |
| 3.48 | 25.61 | 31989 |
| 3.46 | 25.71 | 37163 |
| 3.44 | 25.87 | 17863 |
| 3.44 | 25.88 | 93 |
| 3.44 | 25.91 | 19810 |
| 3.43 | 25.93 | 848 |
| 3.43 | 25.95 | 1050 |
| 3.39 | 26.25 | 150655 |
| 3.39 | 26.29 | 24911 |
| 3.39 | 26.29 | 8730 |
| 3.37 | 26.39 | 216 |
| 3.37 | 26.42 | 16285 |
| 3.37 | 26.45 | 210974 |
| 3.36 | 26.47 | 3101 |
| 3.36 | 26.49 | 1017 |
| 3.36 | 26.51 | 53295 |
| 3.36 | 26.52 | 7871 |
| 3.35 | 26.59 | 242045 |
| 3.35 | 26.62 | 2185 |
| 3.34 | 26.64 | 71101 |
| 3.31 | 26.88 | 1704 |
| 3.31 | 26.89 | 2338 |
| 3.31 | 26.91 | 4037 |
| 3.30 | 26.96 | 9009 |
| 3.30 | 26.98 | 11414 |
| 3.30 | 27.00 | 1934 |
| 3.30 | 27.01 | 22733 |
| 3.29 | 27.08 | 23947 |
| 3.27 | 27.24 | 29208 |
| 3.26 | 27.31 | 11427 |
| 3.25 | 27.41 | 3247 |
| 3.25 | 27.44 | 2855 |
| 3.25 | 27.44 | 1462 |
| 3.23 | 27.61 | 598 |
| 3.22 | 27.64 | 17742 |
| 3.22 | 27.69 | 18950 |
| 3.22 | 27.70 | 4627 |
| 3.22 | 27.72 | 9108 |
| 3.21 | 27.74 | 96136 |
| 3.21 | 27.77 | 10757 |
| 3.21 | 27.79 | 176 |
| 3.21 | 27.79 | 47032 |
| 3.20 | 27.82 | 203 |
| 3.20 | 27.85 | 111598 |
| 3.20 | 27.90 | 11180 |
| 3.20 | 27.90 | 60296 |
| 3.19 | 27.92 | 11 |
| 3.19 | 27.97 | 10088 |
| 3.19 | 27.98 | 20562 |
| 3.17 | 28.10 | 8540 |
| 3.17 | 28.14 | 64575 |
| 3.16 | 28.18 | 275 |
| 3.16 | 28.19 | 19068 |
| 3.16 | 28.21 | 2617 |
| 3.16 | 28.22 | 183346 |
| 3.15 | 28.29 | 151985 |
| 3.15 | 28.30 | 15925 |
| 3.15 | 28.30 | 47761 |
| 3.15 | 28.31 | 6299 |
| 3.15 | 28.33 | 166298 |
| 3.13 | 28.47 | 422 |
| 3.13 | 28.50 | 13754 |
| 3.12 | 28.60 | 10200 |
| 3.12 | 28.62 | 3423 |
| 3.11 | 28.70 | 474 |
| 3.11 | 28.70 | 4939 |
| 3.10 | 28.74 | 124426 |
| 3.10 | 28.76 | 119099 |
| 3.09 | 28.87 | 476 |
| 3.09 | 28.90 | 1328 |
| 3.09 | 28.91 | 1693 |
| 3.07 | 29.02 | 21250 |
| 3.07 | 29.04 | 1005 |
| 3.07 | 29.04 | 63578 |
| 3.07 | 29.06 | 1 |
| 3.06 | 29.15 | 5626 |
| 3.06 | 29.19 | 5851 |
| 3.05 | 29.21 | 33 |
| 3.05 | 29.22 | 1795 |
| 3.05 | 29.23 | 145220 |
| 3.05 | 29.25 | 672 |
| 3.05 | 29.28 | 125 |
| 3.04 | 29.32 | 1298 |
| 3.04 | 29.32 | 1 |
| 3.04 | 29.32 | 15898 |
| 3.03 | 29.46 | 177 |
| 3.03 | 29.48 | 139834 |
| 3.03 | 29.50 | 1556 |
| 3.02 | 29.54 | 6659 |
| 3.02 | 29.59 | 270 |
| 3.01 | 29.61 | 9320 |
| 3.01 | 29.67 | 10021 |
| 3.00 | 29.73 | 821 |
| 3.00 | 29.75 | 6801 |
| 3.00 | 29.76 | 1126 |
| 3.00 | 29.77 | 2065 |
| 2.99 | 29.87 | 52283 |
| 2.99 | 29.91 | 8862 |
| 2.98 | 29.93 | 2325 |
| 2.98 | 29.94 | 1785 |
| 2.98 | 29.96 | 34780 |

TABLE 8
5-MeO-T•HCl

| d-spacing (Å) | °2θ ± 0.2°2θ | Intensity |
|---|---|---|
| 14.32 | 6.17 | 314 |
| 7.22 | 12.25 | 1065 |
| 7.16 | 12.35 | 702 |
| 6.29 | 14.07 | 12899 |
| 6.09 | 14.52 | 1825 |
| 5.47 | 16.18 | 30645 |
| 5.44 | 16.28 | 185 |
| 5.11 | 17.34 | 1712 |
| 4.86 | 18.23 | 1392 |
| 4.77 | 18.57 | 6803 |
| 4.77 | 18.57 | 8 |
| 4.45 | 19.93 | 15766 |
| 4.42 | 20.08 | 5542 |
| 4.26 | 20.86 | 727 |
| 4.20 | 21.12 | 1101 |
| 4.18 | 21.23 | 11983 |
| 4.15 | 21.41 | 16238 |
| 4.15 | 21.42 | 7572 |
| 4.10 | 21.65 | 4334 |
| 4.01 | 22.13 | 10 |
| 3.93 | 22.61 | 30907 |
| 3.83 | 23.21 | 2228 |
| 3.82 | 23.24 | 2418 |
| 3.79 | 23.44 | 7083 |
| 3.78 | 23.49 | 56460 |
| 3.62 | 24.57 | 3978 |
| 3.62 | 24.57 | 10955 |
| 3.61 | 24.64 | 17812 |
| 3.58 | 24.84 | 188 |
| 3.56 | 24.98 | 3088 |
| 3.51 | 25.36 | 46220 |
| 3.48 | 25.60 | 1061 |
| 3.33 | 26.78 | 1658 |
| 3.32 | 26.81 | 209 |
| 3.29 | 27.07 | 24853 |
| 3.26 | 27.34 | 1402 |
| 3.23 | 27.62 | 29 |
| 3.17 | 28.13 | 19213 |
| 3.15 | 28.35 | 7496 |
| 3.14 | 28.35 | 6250 |
| 3.13 | 28.54 | 813 |
| 3.06 | 29.20 | 9666 |
| 3.05 | 29.28 | 19783 |
| 3.04 | 29.36 | 14738 |
| 3.01 | 29.65 | 19475 |
| 2.98 | 29.94 | 7719 |

TABLE 9
5-Br-T•HCl

| d-spacing (Å) | °2θ ± 0.2°2θ | Intensity |
|---|---|---|
| 14.59 | 6.05 | 27345 |
| 7.42 | 11.92 | 918 |
| 7.29 | 12.13 | 9469 |
| 6.20 | 14.28 | 22093 |
| 5.82 | 15.22 | 10876 |
| 5.57 | 15.91 | 22762 |
| 5.31 | 16.67 | 27479 |
| 4.86 | 18.23 | 42630 |
| 4.79 | 18.52 | 24844 |
| 4.69 | 18.91 | 132963 |
| 4.63 | 19.16 | 8353 |
| 4.46 | 19.88 | 250821 |
| 4.31 | 20.60 | 42351 |
| 4.29 | 20.67 | 138066 |
| 4.23 | 20.96 | 542 |
| 4.22 | 21.02 | 22563 |
| 4.13 | 21.49 | 261 |
| 4.08 | 21.78 | 9769 |
| 3.96 | 22.41 | 27873 |
| 3.94 | 22.53 | 82675 |
| 3.91 | 22.70 | 131375 |
| 3.88 | 22.90 | 594538 |
| 3.86 | 23.03 | 12570 |
| 3.79 | 23.44 | 157522 |
| 3.78 | 23.51 | 11462 |
| 3.71 | 23.97 | 113431 |
| 3.65 | 24.33 | 3650 |
| 3.65 | 24.39 | 8 |
| 3.63 | 24.49 | 525150 |
| 3.59 | 24.81 | 58892 |
| 3.48 | 25.55 | 87932 |
| 3.45 | 25.81 | 21309 |
| 3.37 | 26.39 | 74041 |
| 3.36 | 26.47 | 10814 |
| 3.36 | 26.52 | 298103 |
| 3.25 | 27.42 | 19076 |
| 3.22 | 27.64 | 44755 |
| 3.17 | 28.11 | 81945 |
| 3.16 | 28.20 | 12499 |
| 3.15 | 28.28 | 405 |
| 3.14 | 28.38 | 215143 |
| 3.11 | 28.64 | 35582 |
| 3.10 | 28.78 | 4482 |
| 3.05 | 29.27 | 5726 |
| 3.02 | 29.60 | 134279 |

TABLE 10
5-Cl-T•HCl

| d-spacing (Å) | °2θ ± 0.2°2θ | Intensity |
|---|---|---|
| 14.10 | 6.26 | 163 |
| 7.35 | 12.04 | 65 |
| 7.05 | 12.54 | 209 |
| 6.23 | 14.21 | 3258 |
| 6.16 | 14.37 | 4276 |
| 5.45 | 16.24 | 926 |
| 5.33 | 16.63 | 26448 |
| 5.20 | 17.04 | 2089 |
| 4.70 | 18.85 | 7480 |
| 4.70 | 18.86 | 3949 |
| 4.51 | 19.65 | 1355 |
| 4.41 | 20.11 | 26092 |
| 4.30 | 20.62 | 13858 |
| 4.27 | 20.77 | 6963 |
| 4.16 | 21.32 | 4617 |
| 4.13 | 21.51 | 25 |
| 4.13 | 21.52 | 11173 |
| 4.12 | 21.57 | 1070 |
| 4.00 | 22.22 | 4928 |
| 3.98 | 22.29 | 1540 |
| 3.93 | 22.63 | 22556 |
| 3.88 | 22.88 | 214 |
| 3.87 | 22.98 | 79861 |
| 3.85 | 23.11 | 2250 |
| 3.67 | 24.21 | 22383 |
| 3.63 | 24.48 | 12895 |
| 3.62 | 24.60 | 18137 |
| 3.59 | 24.76 | 50887 |
| 3.53 | 25.24 | 366 |
| 3.51 | 25.35 | 1316 |
| 3.44 | 25.86 | 6243 |
| 3.39 | 26.26 | 5709 |
| 3.36 | 26.49 | 156 |
| 3.26 | 27.31 | 1 |
| 3.26 | 27.32 | 31294 |
| 3.24 | 27.51 | 543 |
| 3.19 | 27.94 | 28 |
| 3.17 | 28.08 | 19198 |
| 3.17 | 28.09 | 9879 |
| 3.15 | 28.27 | 2514 |
| 3.11 | 28.64 | 4197 |
| 3.08 | 28.96 | 9942 |
| 3.05 | 29.25 | 24450 |

TABLE 11
5-F-T•HCl

| d-spacing (Å) | °2θ ± 0.2°2θ | Intensity |
|---|---|---|
| 12.84 | 6.88 | 210 |
| 7.19 | 12.31 | 788 |
| 6.42 | 13.78 | 3611 |
| 6.26 | 14.13 | 26974 |
| 5.77 | 15.35 | 22687 |
| 5.16 | 17.17 | 3783 |
| 5.15 | 17.19 | 117642 |
| 4.83 | 18.34 | 20675 |
| 4.75 | 18.66 | 1455 |
| 4.55 | 19.48 | 5574 |
| 4.52 | 19.61 | 42959 |
| 4.34 | 20.47 | 21555 |
| 4.28 | 20.73 | 22939 |
| 4.21 | 21.09 | 14452 |
| 4.17 | 21.31 | 11121 |
| 4.11 | 21.62 | 35 |
| 4.02 | 22.10 | 9898 |
| 4.01 | 22.14 | 150 |
| 3.96 | 22.46 | 89902 |
| 3.91 | 22.72 | 118920 |
| 3.86 | 23.01 | 3327 |
| 3.84 | 23.15 | 84956 |
| 3.79 | 23.47 | 163672 |
| 3.78 | 23.51 | 17989 |
| 3.59 | 24.76 | 95094 |
| 3.59 | 24.77 | 172160 |
| 3.57 | 24.94 | 14452 |
| 3.53 | 25.22 | 13 |
| 3.52 | 25.28 | 15628 |
| 3.37 | 26.44 | 22190 |
| 3.26 | 27.32 | 283 |
| 3.23 | 27.61 | 41452 |
| 3.21 | 27.76 | 366 |
| 3.20 | 27.81 | 7405 |
| 3.20 | 27.84 | 167 |
| 3.19 | 27.95 | 754 |
| 3.13 | 28.45 | 12041 |
| 3.13 | 28.49 | 2348 |
| 3.05 | 29.29 | 19005 |
| 3.02 | 29.55 | 193115 |
| 3.01 | 29.65 | 74033 |
| 3.01 | 29.69 | 43994 |
| 3.00 | 29.75 | 64780 |

TABLE 12
5-Me-T•HCl

| d-spacing (Å) | °2θ ± 0.2°2θ | Intensity |
|---|---|---|
| 14.28 | 6.18 | 1416 |
| 7.26 | 12.19 | 915 |
| 7.14 | 12.39 | 1897 |
| 6.18 | 14.33 | 4289 |
| 6.15 | 14.38 | 7226 |
| 5.45 | 16.26 | 87 |
| 5.28 | 16.76 | 37258 |
| 5.24 | 16.90 | 4903 |
| 4.77 | 18.59 | 2784 |
| 4.76 | 18.63 | 3461 |
| 4.54 | 19.53 | 4 |
| 4.51 | 19.69 | 15552 |
| 4.25 | 20.86 | 1257 |
| 4.22 | 21.05 | 747 |
| 4.21 | 21.07 | 17033 |
| 4.15 | 21.39 | 3 |
| 4.14 | 21.42 | 20394 |
| 4.04 | 21.98 | 1621 |
| 4.00 | 22.22 | 8803 |
| 3.99 | 22.26 | 247 |
| 3.97 | 22.36 | 14772 |
| 3.94 | 22.54 | 375 |
| 3.82 | 23.25 | 2419 |
| 3.82 | 23.29 | 52088 |
| 3.63 | 24.51 | 18516 |
| 3.61 | 24.66 | 8460 |
| 3.58 | 24.86 | 5055 |
| 3.57 | 24.92 | 5858 |
| 3.57 | 24.92 | 17 |
| 3.57 | 24.95 | 27958 |
| 3.44 | 25.87 | 1806 |
| 3.41 | 26.09 | 3994 |
| 3.39 | 26.25 | 7084 |
| 3.35 | 26.62 | 15 |
| 3.29 | 27.11 | 18945 |
| 3.20 | 27.83 | 2110 |
| 3.19 | 27.98 | 1191 |
| 3.16 | 28.24 | 24554 |
| 3.15 | 28.26 | 11457 |
| 3.15 | 28.34 | 156 |
| 3.11 | 28.68 | 13035 |
| 3.09 | 28.88 | 3801 |
| 3.08 | 28.99 | 6446 |

TABLE 13
6-F-T•HCl

| d-spacing (Å) | °2θ ± 0.2°2θ | Intensity |
|---|---|---|
| 12.19 | 7.25 | 535 |
| 6.89 | 12.83 | 1403 |
| 6.28 | 14.09 | 33003 |
| 6.10 | 14.52 | 1369 |
| 5.74 | 15.43 | 26504 |
| 5.17 | 17.12 | 6021 |
| 5.08 | 17.45 | 161188 |
| 5.06 | 17.51 | 24324 |
| 4.92 | 18.00 | 1907 |
| 4.76 | 18.61 | 14247 |
| 4.45 | 19.95 | 23 |
| 4.37 | 20.33 | 4317 |
| 4.33 | 20.50 | 12087 |
| 4.18 | 21.25 | 15555 |
| 4.14 | 21.45 | 3904 |
| 4.06 | 21.85 | 88846 |
| 3.95 | 22.47 | 13639 |
| 3.94 | 22.52 | 27 |
| 3.90 | 22.78 | 123 |
| 3.87 | 22.93 | 95787 |
| 3.87 | 22.97 | 55740 |
| 3.83 | 23.23 | 34956 |
| 3.69 | 24.08 | 26440 |
| 3.65 | 24.34 | 81295 |
| 3.57 | 24.94 | 10880 |
| 3.55 | 25.07 | 4441 |
| 3.50 | 25.45 | 157044 |
| 3.45 | 25.83 | 92322 |
| 3.45 | 25.83 | 16725 |
| 3.27 | 27.25 | 24543 |
| 3.27 | 27.28 | 6985 |
| 3.25 | 27.41 | 50859 |
| 3.22 | 27.66 | 31087 |
| 3.20 | 27.89 | 428 |
| 3.16 | 28.20 | 111976 |
| 3.14 | 28.39 | 0 |
| 3.08 | 28.92 | 77 |
| 3.07 | 29.06 | 176 |
| 3.05 | 29.28 | 555 |
| 3.03 | 29.42 | 43064 |
| 3.02 | 29.57 | 275734 |
| 2.99 | 29.91 | 13114 |

TABLE 14
7-Me-T•HCl

| d-spacing (Å) | °2θ ± 0.2°2θ | Intensity |
|---|---|---|
| 13.76 | 6.42 | 9144 |
| 7.64 | 11.57 | 7752 |
| 6.88 | 12.86 | 8289 |
| 6.37 | 13.90 | 1238 |
| 5.91 | 14.98 | 192347 |
| 5.51 | 16.08 | 2882 |
| 5.33 | 16.63 | 9324 |
| 4.74 | 18.70 | 136184 |
| 4.66 | 19.02 | 6730 |
| 4.60 | 19.29 | 162 |
| 4.59 | 19.30 | 2743 |
| 4.59 | 19.34 | 1007 |
| 4.42 | 20.09 | 1138 |
| 4.36 | 20.36 | 8061 |
| 4.21 | 21.07 | 97273 |
| 4.16 | 21.36 | 61410 |
| 4.12 | 21.54 | 26422 |
| 4.11 | 21.60 | 60 |
| 4.10 | 21.64 | 107 |
| 4.08 | 21.79 | 24388 |
| 3.98 | 22.32 | 58733 |
| 3.95 | 22.50 | 2697 |
| 3.86 | 23.02 | 53182 |
| 3.82 | 23.26 | 3671 |
| 3.79 | 23.47 | 29116 |
| 3.76 | 23.65 | 6772 |
| 3.76 | 23.67 | 30285 |
| 3.56 | 25.00 | 202100 |
| 3.56 | 25.01 | 7436 |
| 3.54 | 25.17 | 192990 |
| 3.44 | 25.88 | 311 |
| 3.37 | 26.43 | 725 |
| 3.32 | 26.85 | 42092 |
| 3.30 | 27.01 | 75678 |
| 3.27 | 27.23 | 30891 |
| 3.27 | 27.25 | 29431 |
| 3.25 | 27.42 | 10 |
| 3.25 | 27.46 | 1343 |
| 3.22 | 27.67 | 54424 |
| 3.18 | 28.00 | 107 |
| 3.08 | 28.94 | 61941 |
| 3.08 | 28.96 | 479 |
| 3.07 | 29.11 | 38729 |
| 3.04 | 29.31 | 80052 |
| 3.01 | 29.70 | 7759 |
| 2.99 | 29.86 | 6748 |

### REFERENCES

Berger, M. L., Palangsuntikul, R., Rebernik, P., Wolschann, P. & Berner, H. (2012). Curr. Med. Chem. 19, 3044-3057.
Bruker (2018). APEX3, SAINT, and SADABS. Bruker AXS Inc., Madison, Wisconsin, USA.
Chang, A. S.-S., Chang, S. M. & Starnes, D. M. (1993). Eur. J. Pharmacol. 247, 239-248.
Dolomanov, O. V., Bourhis, L. J., Gildea, R. J., Howard, J. A. K. & Puschmann, H. (2009). J. Appl. Cryst. 42, 339-341.
Peroutka, S. J., McCarthy, B. G. & Guan, X.-M. (1991). Life Sci. 49, 409-418.
Sheldrick, G. M. (2015a). Acta Cryst. A 71, 3-8.
Sheldrick, G. M. (2015b). Acta Cryst. C71, 3-8.
Westrip, S. P. (2010). J. Appl. Cryst. 43, 920-925.

1-24. (canceled)

25. A crystalline form of a substituted tryptammonium hydrochloride salt selected from the group consisting of:
crystalline form 1 of 2-(7-methyl-1H-indol-3-yl)ethan-1-aminium chloride (1-methyltryptammonium chloride);
crystalline form 1 of 2-(2-methyl-1-phenyl-1H-indol-3-yl)ethan-1-aminium chloride (1-phenyl-2-methyltryptammonium chloride);
crystalline form 1 of 2-(5-methoxy-1H-indol-3-yl)ethan-1-aminium chloride (5-methoxytryptammonium chloride);
crystalline form 1 of 2-(5-bromo-1H-indol-3-yl)ethylazanium chloride (5-bromotryptammonium chloride);
crystalline form 1 of 2-(5-chloro-1H-indol-3-yl)ethylazanium chloride (5-chlorotryptammonium chloride);
crystalline form 1 of 2-(5-fluoro-1H-indol-3-yl)ethan-1-aminium chloride (5-fluorotryptammonium chloride);
crystalline form 1 of 2-(5-methyl-1H-indol-3-yl)ethylazanium chloride (5-methyltryptammonium chloride);
crystalline form 1 of 2-(6-fluoro-1H-indol-3-yl)ethan-1-aminium chloride (6-fluorotryptammonium chloride); and
crystalline form 1 of 2-(7-methyl-1H-indol-3-yl)ethyl]azanium chloride (7-methyltryptammonium chloride).

26. The crystalline form according to claim 25, wherein
crystalline form 1 of 1-methyltryptammonium chloride is characterized by at least one of:
a trigonal, R3c space group at a temperature of about 297 K;
unit cell dimensions a=29.3337(13), b=29.3337(13), c=7.3922(6), a=90°, β=90°, and γ=120°;
an x-ray powder diffraction (XRPD) pattern substantially similar to FIG. 6; and
an XRPD pattern characterized by at least two peaks selected from 6.0, 10.4, 15.1, 16.0, and 19.4 ° 2θ±0.2 ° 2θ,
crystalline form 1 of 1-phenyl-2-methyltryptammonium chloride is characterized by at least one of:
a monoclinic, P2_{1/n} space group at a temperature of about 297 K;
unit cell dimensions a=10.3990(6), b=16.3016(10), c=37.091(2), α=90°, β=97.963(2°), and γ=90°;
an x-ray powder diffraction (XRPD) pattern substantially similar to FIG. 7; and
an XRPD pattern characterized by at least two peaks selected from 9.0, 15.4, and 17.2 ° 2θ,
crystalline form 1 of 5-methoxytryptammonium chloride is characterized by at least one of:
a monoclinic, P2_{1/C} space group at a temperature of about 297 K;
unit cell dimensions a=14.6858(8), b=8.3613(4), c=9.7878(5), α=90°, β=102.742(2°), and γ=90°;
an x-ray powder diffraction (XRPD) pattern substantially similar to FIG. 8; and
an XRPD pattern characterized by at least two peaks selected from 14.1, 16.2, and 22.6 ° 2θ±0.2 ° 2θ,
crystalline form 1 of 5-bromotryptammonium chloride is characterized by at least one of:
an orthorhombic, Pbca space group at a temperature of about 297 K;
unit cell dimensions a=8.6153(6), b=9.3766(5), c=29.173(2), α=90°, β=90°, and γ=90°;
an x-ray powder diffraction (XRPD) pattern substantially similar to FIG. 9; and
an XRPD pattern characterized by at least two peaks selected from 6.1, 16.7, and 19.9 ° 2θ±0.2 ° 2θ,
crystalline form 1 of 5-chlorotryptammonium chloride is characterized by at least one of:
a monoclinic, P2_{1/C} space group at a temperature of about 297 K;
unit cell dimensions a=14.7030(9), b=8.6058(5), c=9.4141(5), a=90°, β=106.450(2°), and γ=90°;
an x-ray powder diffraction (XRPD) pattern substantially similar to FIG. 10; and
an XRPD pattern characterized by at least two peaks selected from 16.6, 20.1, and 23.0 ° 2θ±0.2 ° 2θ,
crystalline form 1 of 5-fluorotryptammonium chloride is characterized by at least one of:
an orthorhombic, Pbca space group at a temperature of about 297 K;
unit cell dimensions a=8.6708(4), b=9.6684(5), c=25.6854(12), α=90°, β=90°, and γ=90°;
an x-ray powder diffraction (XRPD) pattern substantially similar to FIG. 11; and
an XRPD pattern characterized by at least two peaks selected from 14.1, 15.3, and 17.2 ° 2θ±0.2 ° 2θ,
crystalline form 1 of 5-methyltryptammonium chloride is characterized by at least one of:
a monoclinic, P2_{1/C} space group at a temperature of about 297 K;
unit cell dimensions a=14.9939(10), b=8.4270(5), c=9.5388(6), α=90°, β=107.774(2°), and γ=90°;
an x-ray powder diffraction (XRPD) pattern substantially similar to FIG. 12; and
an XRPD pattern characterized by at least two peaks selected from 6.2, 19.7, and 23.3 ° 2θ±0.2 ° 2θ,
crystalline form 1 of 6-fluorotryptammonium chloride is characterized by at least one of:
an orthorhombic, Pbca space group at a temperature of about 297 K;
unit cell dimensions a=8.3572(4), b=10.3493(5), c=24.3824(13), α=90°, β=90°, and γ=90°;
an x-ray powder diffraction (XRPD) pattern substantially similar to FIG. 13; and
an XRPD pattern characterized by at least two peaks selected from 14.1, 15.4, and 21.9 ° 2θ±0.2 ° 2θ, and
crystalline form 1 of 7-methyltryptammonium chloride is characterized by at least one of:
an orthorhombic, Pbca space group at a temperature of about 297 K;
unit cell dimensions a=9.1893(5), b=9.3259(4), c=27.5149(15), α=90°, β=90°, and γ=90°;
an x-ray powder diffraction (XRPD) pattern substantially similar to FIG. 14; and
an XRPD pattern characterized by at least two peaks selected from 6.4, 15.0, and 18.7 ° 2θ±0.2 ° 2θ.

27. A composition comprising a crystalline substituted tryptammonium hydrochloride salt according to claim 25 and an excipient.

28. A composition comprising a crystalline substituted tryptammonium hydrochloride salt according to claim 25 as a first component and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone.

29. A composition comprising one or more crystalline substituted tryptammonium hydrochloride salts according to claim 25 as a first component and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone.

30. The composition according to claim 29 further comprising an excipient.

31. A composition comprising a crystalline substituted tryptammonium hydrochloride salt according to claim 26 and an excipient.

32. A composition comprising a crystalline substituted tryptammonium hydrochloride salt according to claim 26 as a first component and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone.

33. A composition comprising one or more crystalline substituted tryptammonium hydrochloride salts according to claim 26 as a first component and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone.

34. The composition according to claim 33 further comprising an excipient.
